SEQUENCE LISTING (46 sequences)

SEQ ID NO: 1 - protein, 122 aa, synthetic construct
DVQVQESGPDLVKPSQSLSLTCTVTGHSITRGSSWHWIRQFPGNKLEWMGYIHYGGGTSYNPSLKSRISITRDTSKNQFFLQLNSVTTEDTATFFCARYSYDITYRWFFDVWGAGTTVIVSS

SEQ ID NO: 2 - protein, 107 aa, synthetic construct
DILLTQSPAILSVSPGERVSFSCRASQNIGTSIHWYQQRKNGSPRLLIKYASESISGIPSRFSGSGSGTDFTLSINSVESEDMADYYCQQNNNWPLTFGAGTKLELK

SEQ ID NO: 3 - protein, 120 aa, synthetic construct
EIQLVQSGAEVKKPGSSVKVSCKASGYTFTDYYINWMRQAPGQGLEWIGWIDPGSGNTKYNEKFKGRATLTVDTSTNTAYMELSSLRSEDTAFYFCAREKTTYYYAMDYWGQGTLVTVSS

SEQ ID NO: 4 - protein, 112 aa, synthetic construct
DIQMTQSPSTLSASVGDRVTITCRSSKSLLHSNGDTFLYWFQQKPGKAPKLLMYRMSNLASGVPSRFSGSGSGTEFTLTISSLQPDDFATYYCMQHLEYPFTFGQGTKVEVK

SEQ ID NO: 5 - protein, 122 aa, synthetic construct
EIQLVQSGAEVKKPGSSVKVSCKASGHSITRGSSWHWMRQAPGQGLEWIGYIHYGGGTSYNPSLKSRATLTVDTSTNTAYMELSSLRSEDTAFYFCARYSYDITYRWFFDVWGQGTLVTVSS

SEQ ID NO: 6 - protein, 107 aa, synthetic construct
DIQMTQSPSTLSASVGDRVTITCRASQNIGTSIHWFQQKPGKAPKLLMYRASESISGVPSRFSGSGSGTEFTLTISSLQPDDFATYYCQQNNNWPLTFGQGTKVEVK

SEQ ID NO: 7 - protein, 122 aa, synthetic construct
EIQLVQSGAEVKKPGSSVKVSCKVTGHSITRGSSWHWMRQAPGQGLEWIGYIHYGGGTSYNPSLKSRATLTVDTSTNTAYMELSSLRSEDTAFYFCARYSYDITYRWFFDVWGQGTLVTVSS

SEQ ID NO: 8 - protein, 122 aa, synthetic construct
EIQLVQSGAEVKKPGSSVKVSCKVTGHSITRGSSWHWMRQAPGQGLEWIGYIHYGGGTSYNPSLKSRITITRDTSTNTAYMELSSLRSEDTAFYFCARYSYDITYRWFFDVWGQGTLVTVSS

SEQ ID NO: 9 - protein, 122 aa, synthetic construct
EIQLVQSGAEVKKPGSSVKVSCKVTGHSITRGSSWHWMRQAPGQGLEWIGYIHYGGGTSYNPSLKSRATLTRDTSTNTAYMELSSLRSEDTAFYFCARYSYDITYRWFFDVWGQGTLVTVSS

SEQ ID NO: 10 - protein, 122 aa, synthetic construct
EIQLVQSGAEVKKPGSSVKVSCKVTGHSITRGSSWHWMRQAPGQGLEWIGYIHYGGGTSYNPSLKSRATLTRDTSTNTFYMELSSLRSEDTAFYFCARYSYDITYRWFFDVWGQGTLVTVSS

SEQ ID NO: 11 - protein, 122 aa, synthetic construct
EIQLVQSGAEVKKPGSSVKVSCKVTGHSITRGSSWHWMRQAPGQGLEWIGYIHYGGGTSYNPSLKSRITITRDTSTNTFYMELSSLRSEDTAFYFCARYSYDITYRWFFDVWGQGTLVTVSS

SEQ ID NO: 12 - protein, 107 aa, synthetic construct
DIQMTQSPSTLSASVGDRVTITCRASQNIGTSIHWFQQKPGKAPKLLIKRASESISGVPSRFSGSGSGTEFTLTISSLQPDDFATYYCQQNNNWPLTFGQGTKVEVK

SEQ ID NO: 13 - protein, 107 aa, synthetic construct
DIQMTQSPSTLSASVGDRVTITCRASQNIGTSIHWYQQKPGKAPKLLIKRASESISGVPSRFSGSGSGTEFTLTISSLQPDDFATYYCQQNNNWPLTFGQGTKVEVK

SEQ ID NO: 14 - protein, 107 aa, synthetic construct
DIQMTQSPSTLSASVGDRVTITCRASQNIGTSIHWYQQKPGKAPKLLIKYASESISGVPSRFSGSGSGTEFTLTISSLQPDDFATYYCQQNNNWPLTFGQGTKVEVK

SEQ ID NO: 15 - protein, 107 aa, synthetic construct
DIQMTQSPSTLSASVGDRVTITCRASQNIGTSIHWYQQKPGKAPKLLIKYASESISGVPSRFSGSGSGTEFTLTISSLQPDDFATYYCQQNNNWPLTFGQGTKVEIK

SEQ ID NO: 16 - DNA, 19 bp, synthetic construct
  source 1..19: mol_type = other DNA
cgccaccggcgcgcattct

SEQ ID NO: 17 - DNA, 27 bp, synthetic construct
  source 1..27: mol_type = other DNA
ggtgctagcgctggacacggtcaccag

SEQ ID NO: 18 - DNA, 36 bp, synthetic construct
  source 1..36: mol_type = other DNA
cacaggcgcgcactccgacatccagatgacacagag

SEQ ID NO: 19 - DNA, 30 bp, synthetic construct
  source 1..30: mol_type = other DNA
gccaccgtacgcttcacctccaccttggtg

SEQ ID NO: 20 - DNA, 33 bp, synthetic construct
  source 1..33: mol_type = other DNA
gtcttgcaaggtgacaggccactccatcacaag

SEQ ID NO: 21 - DNA, 18 bp, synthetic construct
  source 1..18: mol_type = other DNA
accttcacgctggagcct

SEQ ID NO: 22 - DNA, 28 bp, synthetic construct
  source 1..28: mol_type = other DNA
cacaagggacaccagcacaaacaccgct

SEQ ID NO: 23 - DNA, 33 bp, synthetic construct
  source 1..33: mol_type = other DNA
atggtgatcctagacttcaggcttggattgtag

SEQ ID NO: 24 - DNA, 24 bp, synthetic construct
  source 1..24: mol_type = other DNA
caccctgacaagggacaccagcac

SEQ ID NO: 25 - DNA, 18 bp, synthetic construct
  source 1..18: mol_type = other DNA
gccctagacttcaggctt

SEQ ID NO: 26 - DNA, 30 bp, synthetic construct
  source 1..30: mol_type = other DNA
cacaaacaccttttatatggagctgagctc

SEQ ID NO: 27 - DNA, 18 bp, synthetic construct
  source 1..18: mol_type = other DNA
ctggtgtcccttgtcagg

SEQ ID NO: 28 - DNA, 19 bp, synthetic construct
  source 1..19: mol_type = other DNA
ctggtgtcccttgtgatgg

SEQ ID NO: 29 - DNA, 37 bp, synthetic construct
  source 1..37: mol_type = other DNA
taagctgctgatcaagagagcttctgagtccatcagc

SEQ ID NO: 30 - DNA, 18 bp, synthetic construct
  source 1..18: mol_type = other DNA
ggggccttgccgggcttc

SEQ ID NO: 31 - DNA, 23 bp, synthetic construct
  source 1..23: mol_type = other DNA
catccattggtaccagcagaagc

SEQ ID NO: 32 - DNA, 18 bp, synthetic construct
  source 1..18: mol_type = other DNA
ctggtgccgatattctgg

SEQ ID NO: 33 - DNA, 33 bp, synthetic construct
  source 1..33: mol_type = other DNA
gctgatcaagtacgcttctgagtccatcagcgg

SEQ ID NO: 34 - DNA, 18 bp, synthetic construct
  source 1..18: mol_type = other DNA
agcttaggggccttgccg

SEQ ID NO: 35 - DNA, 32 bp, synthetic construct
  source 1..32: mol_type = other DNA
caaggtggagatcaagcgtacggtggctgcac

SEQ ID NO: 36 - DNA, 21 bp, synthetic construct
  source 1..21: mol_type = other DNA
gtgccctggccaaatgtcagg

SEQ ID NO: 37 - protein, 452 aa, synthetic construct
EIQLVQSGAEVKKPGSSVKVSCKVTGHSITRGSSWHWMRQAPGQGLEWIGYIHYGGGTSYNPSLKSRITITRDTSTNTFYMELSSLRSEDTAFYFCARYSYDITYRWFFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, synthetic construct
DIQMTQSPSTLSASVGDRVTITCRASQNIGTSIHWYQQKPGKAPKLLIKYASESISGVPSRFSGSGSGTEFTLTISSLQPDDFATYYCQQNNNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 107 aa, synthetic construct [sequence omitted]

SEQ ID NO: 41 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 42 - protein, 140 aa, synthetic construct
MRVLILLCLFTAFPGVLSDVQVQESGPDLVKPSQSLSLTCTVTGHSITRGSSWHWIRQFPGNKLEWMGYIHYGGGTSYNPSLKSRISITRDTSKNQFFLQLNSVTTEDTATFFCARYSYDITYRWFFDVWGAGTTVIVSS

SEQ ID NO: 43 - protein, 127 aa, synthetic construct
MVSTPQFLVFLLFWIPASRGDILLTQSPAILSVSPGERVSFSCRASQNIGTSIHWYQQRKNGSPRLLIKYASESISGIPSRFSGSGSGTDFTLSINSVESEDMADYYCQQNNNWPLTFGAGTKLELK

SEQ ID NO: 44 - DNA, 33 bp, synthetic construct
  source 1..33: mol_type = other DNA
gtcttgcaaggcttctggccactccatcacaag

SEQ ID NO: 45 - protein, 122 aa, synthetic construct
EIQLVQSGAEVKKPGSSVKVSCKASGHSITRGSSWHWMRQAPGQGLEWIGYIHYGGGTSYNPSLKSRATLTRDTSTNTFYMELSSLRSEDTAFYFCARYSYDITYRWFFDVWGQGTLVTVSS

SEQ ID NO: 46 - protein, 122 aa, synthetic construct
EIQLVQSGAEVKKPGSSVKVSCKASGHSITRGSSWHWMRQAPGQGLEWIGYIHYGGGTSYNPSLKSRITITRDTSTNTFYMELSSLRSEDTAFYFCARYSYDITYRWFFDVWGQGTLVTVSS

(19) United States
(12) Patent Application Publication
(10) Pub. No.: US 2025/0041437 A1
(43) Pub. Date: Feb. 6, 2025

(54) HUMANIZED MUC1 ANTIBODY AND ANTIBODY DRUG CONJUGATE

(71) Applicant: Sun Pharma Advanced Research Company Limited, Mumbai (IN)

(72) Inventors: Nitin Krishnaji DAMLE, Mumbai (IN); Vikas B. MEHRA, Mahendragarh (IN); Adaitya Prasad BEHERA, Khorda (IN); Shruti GUPTA, Ahmedabad (IN); Vinod P KUMAR, Vadodara (IN); Akhil THAKER, Rajkot (IN); Ragithakumari CHRUVATTIL, Tapi (IN); Argha DAS, Kolkata (IN); Biswajit SAMANTA, Vadodara (IN)

(21) Appl. No.: 18/790,601

(22) Filed: Jul. 31, 2024

(30) Foreign Application Priority Data
Jul. 31, 2023 (IN) 202321051338

(51) Int. Cl. A61K 47/68; A61P 35/00; C07K 16/30

(52) U.S. Cl. CPC A61K 47/6851; A61K 47/68031; A61P 35/00; C07K 16/3092; C07K 2317/24; C07K 2317/73; C07K 2317/94

(57) ABSTRACT

This invention relates to humanized MUC1 antibodies, antibody drug conjugates (ADCs) comprising such antibodies, and their use in treating disorders, such as cancer, especially where MUC1 is overexpressed.

This application claims the benefit of Indian Patent Application No. 202321051338, filed Jul. 31, 2023, which is hereby incorporated by reference.

A computer readable form of the Sequence Listing is filed with this application by electronic submission and is incorporated into this application by reference in its entirety. The ST26 Sequence Listing is contained in the file created on Jul. 31, 2024 having the file name “159818-02601_SL.xml”.

### FIELD OF THE INVENTION

This invention relates to humanized MUC1 antibodies, antibody drug conjugates (ADCs) comprising such antibodies, and their use in treating disorders, such as cancer, especially where MUC1 is overexpressed.

### BACKGROUND OF THE INVENTION

The MUC1 glycoprotein is over-expressed by a variety of high-incidence, high-mortality human epithelial malignancies, including breast, prostate, pancreas, ovarian, lung, and colon carcinomas, as well as by the malignant plasma cells of multiple myeloma, and by the myelogenous cells of acute myelogenous leukemia. Because of its preferentially high expression by malignant cells, and because it is expressed on the cell surface and therefore is an exposed molecule, MUC1 has been studied as both a target for directed cancer therapy and as a marker of disease progression. Rivalland, et al., Expert Opin Biol Ther, 15 (2015) 1773-1787; Taylor-Papadimitriou et al., Biochem Soc Trans, 46 (2018) 659-668.

Structurally, the MUC1 molecule is a transmembrane glycoprotein (termed MUC-TM). MUC-TM is a heterodimer consisting of an extracellular domain containing between 20 to 125 repeats or more of a 20 amino acid-long sequence (termed the variable number tandem repeat, VNTR), a transmembrane domain, and a short cytoplasmic tail mediating intracellular signaling. The MUC1 molecule is auto-proteolytically cleaved within the SEA (Sperm protein, Enterokinase and Agrin) module, a highly-conserved domain of 110 amino acids. This results in a large extracellular α subunit containing the tandem repeat array bound in a strong non-covalent interaction to a transmembrane β subunit containing the transmembrane and cytoplasmic domains of the molecule. Binding of the α chain to the β chain is intermittent: The α chain binds the β chain in an on-and-off manner. While the β chain remains on the cell surface all the time, the α chain with its VNTR remains cell-bound only intermittently.

A number of anti-MUC1 antibodies have been reported in the literature with the great majority directed against the highly immunogenic VNTR of the α chain. While anti-VNTR antibodies can successfully bind MUC1+ cells in vitro, the shedding of the MUC1 α chain containing the VNTR into the peripheral circulation in vivo severely compromises the ability of anti-VNTR antibodies to clinically affect MUC1 expressing tumors. Shedding of the α chain off the tumor cell surface not only greatly decreases the number of MUC1 targets for anti-α chain antibodies, but additionally, freely circulating MUC1 α chain in vivo in the periphery can bind and neutralize anti-VNTR antibodies or anti-glycosylated-VNTR antibodies thereby limiting their ability to even reach MUC1-expressing tumor.

Antibodies that recognize cancer-specific truncated O-glycoforms of the VNTR, such as the antibodies PankoMab-Gex, 5E5, SM3, and VU-2-G7, have been proposed as possible ways to overcome the potential toxicity of targeting MUC1 expressed by normal tissues. However, the limitations of targeting the α-chain VNTR, namely its shedding from the cell surface and its ability to bind therapeutically administered anti-MUC1 antibody, remain. Burchell, et al., J Mammary Gland Biol Neoplasia, 6 (2001) 355-364; Fiedler, et al., Eur J Cancer, 63 (2016) 55-63; Ryuko et al., Tumour Biol, 21 (2000) 197-210; Tarp, et al., Glycobiology, 17 (2007) 197-209; Zhou, et al., Molecules, 23 (6) (2018) 1326.

Because of the instability of their target, which, as noted, binds the tumor cell only intermittently in an on-and off mechanism, no anti-MUC1 VNTR antibody has yet been proven to be clinically effective. Wu et al., Cancer Cell International (2022) 22:417; Fiedler, supra; Kimura, et al., Expert Opin Biol Ther, 13 (2013) 35-49; Ibrahim, et al., Clin Cancer Res, 17 (2011) 6822-6830.

In contrast to the α chain and its VNTR, the MUC1 SEA domain formed by the interaction of the α-subunit with the extracellular portion of the β-subunit is a stable cell membrane-fixed molecular moiety. Anti-MUC1 α/β junction antibodies were disclosed in Rubinstein, et al., Cancer Res, 66 (2006) 11247-11253; Pichinuk, et al., Cancer Res, 72 (2012) 3324-3336; Rubinstein, et al., Int J Cancer, 124 (2009) 46-54.

U.S. Pat. No. 8,648,172 discloses certain antibodies that concurrently bind to both the α-subunit and the β-subunit of an intact MUC1 protein (“anti-MUC1 α/β antibodies” which bind to the MUC1 α/β subunit junction but does not substantially bind to either the MUC1 α-subunit or the MUC1 β-subunit in the absence of the other).

International Publication No. WO 2021/186427 discloses certain isolated monoclonal antibodies specifically directed against the junction of the alpha and beta chains (which comprises the SEA domain) of MUC1.

Singh et al., J. Pharmacokinet Pharmacodyn, disclose characterization of the bystander effect of antibody-drug conjugates, such as ADC trastuzumab-vc-MMAE.

There is a continuing need for improved MUC1 antibodies and MUC1 antibody treatments for cancer.

### SUMMARY OF THE INVENTION

The present inventors have developed humanized monoclonal antibodies which bind to the MUC1 SEA domain.

One embodiment is a humanized antibody that binds to an epitope in the MUC1 SEA domain, preferably with a binding affinity KD less than 1000 pM (such as less than 500 pM, less than 100 pM, or less than 30 pM). In one embodiment, the humanized antibody binds to an epitope in the SEA domain of MUC1 (SEQ ID NO: 41) formed preferentially from arginine at position 1108, glutamic acid at position 1109, asparagine at position 1113 and glutamic acid at position 1118 of MUC1. In another embodiment, the humanized antibody binds to the same epitope as a chimeric antibody (e.g., chimeric antibody 5F3) comprising a heavy chain variable region and a light chain variable region comprising the amino acid sequences of SEQ ID NOs: 1 and 2, respectively. In another embodiment, the humanized antibody binds to the same epitope as chimeric antibody 5F3 where the sequences of the heavy and light chains are as provided in SEQ ID NOs: 42 and 43, respectively. In yet another embodiment, the antibody is a humanized form of any one of chimeric antibodies DMB4F4 (4F4), DMB7F3 (7F3), or DMB10F10 (10F10), each of which is described in International Publication No. WO 2021/186427, which is hereby incorporated by reference. In yet another embodiment, the humanized antibody binds to the same epitope as chimeric antibody 4F4, 7F3, or 10F10.

Another embodiment is a humanized antibody comprising a means for binding to an epitope in the SEA domain of MUC1 (SEQ ID NO: 41) formed from arginine at position 1108, glutamic acid at position 1109, asparagine at position 1113 and glutamic acid at position 1118 of MUC1.

Yet another embodiment is a humanized antibody that binds to the same epitope as a chimeric antibody (e.g., chimeric antibody 5F3) comprising a heavy chain variable region and a light chain variable region comprising the amino acid sequences of SEQ ID NOs: 1 and 2, respectively. In another embodiment, the humanized antibody binds to the same epitope as chimeric antibody 5F3 where the sequences of the heavy and light chains are as provided in SEQ ID NOs: 42 and 43, respectively.

Yet another embodiment is a humanized monoclonal antibody which binds to the MUC1 SEA domain, where the antibody comprises:
(a) a heavy chain variable region comprising the amino acid sequence of any one of SEQ ID NOs. 7-11, 45, and 46; and
(b) a light chain variable region comprising the amino acid sequence of any one of SEQ ID NOs. 12-15.

Yet another embodiment is a humanized monoclonal antibody which binds to the MUC1 SEA domain, where the antibody comprises:
(a) a heavy chain variable region comprising the amino acid sequence of any one of SEQ ID NOs. 7-11; and
(b) a light chain variable region comprising the amino acid sequence of any one of SEQ ID NOs. 12-15.

In one embodiment, the humanized antibody comprises:
(a) a heavy chain variable region comprising the amino acid sequence of SEQ ID NO: 8 and a light chain variable region comprising the amino acid sequence of SEQ ID NO: 12;
(b) a heavy chain variable region comprising the amino acid sequence of SEQ ID NO: 8 and a light chain variable region comprising the amino acid sequence of SEQ ID NO: 13;
(c) a heavy chain variable region comprising the amino acid sequence of SEQ ID NO: 8 and a light chain variable region comprising the amino acid sequence of SEQ ID NO: 14;
(d) a heavy chain variable region comprising the amino acid sequence of SEQ ID NO: 9 and a light chain variable region comprising the amino acid sequence of SEQ ID NO: 13;
(e) a heavy chain variable region comprising the amino acid sequence of SEQ ID NO: 9 and a light chain variable region comprising the amino acid sequence of SEQ ID NO: 14;
(f) a heavy chain variable region comprising the amino acid sequence of SEQ ID NO: 10 and a light chain variable region comprising the amino acid sequence of SEQ ID NO: 13;
(g) a heavy chain variable region comprising the amino acid sequence of SEQ ID NO: 10 and a light chain variable region comprising the amino acid sequence of SEQ ID NO: 14;
(h) a heavy chain variable region comprising the amino acid sequence of SEQ ID NO: 11 and a light chain variable region comprising the amino acid sequence of SEQ ID NO: 13;
(i) a heavy chain variable region comprising the amino acid sequence of SEQ ID NO: 11 and a light chain variable region comprising the amino acid sequence of SEQ ID NO: 14; or
(j) a heavy chain variable region comprising the amino acid sequence of SEQ ID NO: 11 and a light chain variable region comprising the amino acid sequence of SEQ ID NO: 15.

In one preferred embodiment, the humanized monoclonal antibody which binds to the MUC1 SEA domain comprises a heavy chain variable region comprising the amino acid sequence of SEQ ID NO: 11 and a light chain variable region comprising the amino acid sequence of SEQ ID NO: 15.

In one embodiment of any of the humanized antibodies described herein, the constant domain of the heavy chain of the humanized antibody comprises the amino acid sequence of SEQ ID NO: 39. In another embodiment of any of the humanized antibodies described herein, the constant domain of the light chain of the humanized antibody comprises the amino acid sequence of SEQ ID NO: 40. In yet another embodiment of any of the humanized antibodies described herein, the constant domain of the heavy chain of the humanized antibody comprises the amino acid sequence of SEQ ID NO: 39 and the constant domain of the light chain of the humanized antibody comprises the amino acid sequence of SEQ ID NO: 40.

In one embodiment of any of the humanized antibodies described herein, of the humanized antibody comprises a heavy chain comprising the amino acid sequence of SEQ ID NO: 37 and a light chain comprising the amino acid sequence of SEQ ID NO: 38.

In one embodiment, the antigen-binding fragment thereof is Fv, single chain Fv (scFv), single chain Fv-Fc (scFv-Fc), Fab′, Fab, F(ab′)₂ or F(ab)₂.

Another embodiment is a nucleic acid molecule (e.g., an isolated nucleic acid molecule) comprising a nucleotide sequence encoding a monoclonal antibody or antigen-binding fragment thereof as described herein.

Yet another embodiment is an expression vector comprising any nucleic acid molecule (e.g., an isolated nucleic acid molecule) as described herein.

Yet another embodiment is a host cell transfected with any expression vector as described herein.

Yet another embodiment is an immunoconjugate comprising any antibody (e.g., an isolated antibody) as described herein and an additional cytotoxic or therapeutic agent. In one embodiment, the cytotoxic agent is selected from alkylating drugs, anthracyclines, pyrimidine derivatives, vinca alkaloids, photodynamic drugs, platinum-containing compounds, taxanes, topoisomerase inhibitors, ribosome inactivating agents, agents that induce DNA damage, tubulin inhibitors, anti-mitotic agents, radioisotopes, cytotoxic antibodies and bacterial toxins. In another embodiment, the cytotoxic agent is pseudomonas exotoxin. In yet another embodiment, the cytotoxic agent is monomethyl auristatin E (MMAE).

In one embodiment, the antibody is conjugated to maleimidocaproyl-valyl-citrullinyl-p-aminobenzyloxycarbonyl also wrote as maleimidocaproyl-Val-Cit-PABC-MMAE (where PABC is p-aminobenzyloxycarbonyl) or mc-vc-PABC-MMAE. Another embodiment is an immunoconjugate comprising a humanized monoclonal antibody which binds to the MUC1 SEA domain, where (i) the antibody comprises a heavy chain variable region comprising the amino acid sequence of SEQ ID NO: 11 and a light chain variable region comprising the amino acid sequence of SEQ ID NO: 15, and (ii) the antibody is conjugated to maleimidocaproyl-Val-Cit-PABC-MMAE.

Another embodiment is an immunoconjugate comprising an antibody as described herein conjugated to maleimidocaproyl-Val-Cit-PABC-MMAE, where the antibody (i) binds to the MUC1 SEA domain and (ii) comprises a heavy chain comprising the amino acid sequence of SEQ ID NO: 37 and a light chain comprising the amino acid sequence of SEQ ID NO: 38.

In one embodiment, the immunoconjugate reduces tumor volume upon administration to a subject with cancer.

In one embodiment, the antibody is bispecific or trispecific. The bispecific antibody may have two different heavy/light chain pairs and two different bindings sites. The bispecific antibody may include a heavy chain variable region described herein (e.g., a heavy chain variable region comprising the amino acid sequence of any one of SEQ ID NOs. 7-11, 45, and 46) and a light chain variable region described herein (e.g., a light chain variable region comprising the amino acid sequence of any one of SEQ ID NOs. 12-15). The bispecific antibody may include an antigen-binding fragment which does not bind to the MUC1 SEA domain.

Yet another embodiment is a pharmaceutical composition comprising (a) a humanized monoclonal antibody as described herein, an immunoconjugate as described herein, and (b) a pharmaceutically acceptable carrier, excipient, or diluent. In one embodiment, the pharmaceutical composition further comprises an additional therapeutic agent.

Yet another embodiment is a method of treatment or amelioration of a disease or disorder comprising administering to a subject in need thereof a therapeutically effective amount of at least one humanized monoclonal antibody as described herein, immunoconjugate as described herein, or a pharmaceutical composition as described herein. In one embodiment, the disease or disorder is cancer, such as a MUC1 expressing cancer. For instance, the cancer may be selected from lung carcinoma, prostate carcinoma, breast carcinoma, ovarian carcinoma, colon carcinoma, pancreatic carcinoma, multiple myeloma, and acute myelogenous leukemia and other cancers expressing MUC1. In another embodiment, the disease or disorder is an autoimmune or an inflammatory disease, such as rheumatoid arthritis, psoriatic arthritis, systemic lupus erythematosus, amyloidosis, or autoimmune pancreatitis. In yet another embodiment, the disease or disorder is a non-malignant clinically significant abnormal growth condition, e.g., cysts, for example renal cysts, thyroid cysts and thyroid masses, or hepatic cysts. In one embodiment, the method further comprises administering to a subject in need thereof an additional therapeutic agent.

Yet another embodiment is a monoclonal antibody as described herein, an immunoconjugate as described herein, or a pharmaceutical composition as described herein for use in a method of treatment or amelioration of a disease or disorder, the method comprising administering to a subject in need thereof a therapeutically effective amount of the monoclonal antibody, the immunoconjugate or the pharmaceutical composition. In one embodiment, the disease or disorder is cancer. In another embodiment, the disease or disorder is a non-malignant abnormal growth condition, such as cysts, for example renal cysts, thyroid cysts and thyroid masses, or hepatic cysts. In another embodiment, the method further comprises administering to a subject in need thereof an additional therapeutic agent.

Yet another embodiment is a method of diagnosing a disease or disorder in a subject, wherein the disease or disorder is associated with MUC-1 expression, the method comprising
(a) contacting a biopsy obtained from the patient with at least one monoclonal antibody of to any one of claims 1-4; and
(b) detecting the monoclonal antibody,
   wherein detection of cells over-expressing MUC1 SEA in the biopsy indicates that the subject is diagnosed with the disease or disorder. In one embodiment, the disease or disorder is cancer. In another embodiment, the disease or disorder is an autoimmune or an inflammatory disease, or wherein said disease or disorder is a non-malignant abnormal growth condition, e.g., cysts, for example renal cysts, thyroid cysts and thyroid masses, or hepatic cysts. In yet another embodiment, the monoclonal antibody is detectably labelled.

Yet another embodiment is a method of imaging of a disease or disorder, the method comprising:
(a) introducing at least one monoclonal antibody as described herein into a subject, wherein the antibody is labelled with a radioisotope or other suitable agent e.g., fluorescent dye, for detection; and
(b) visualizing the detectably-labelled monoclonal antibody,
   wherein detection of cells and/or tissues labelled with the radioisotope or detection agent indicates the presence and/or the location and/or extent and/or the presence of metastases of the disease or disorder in the subject. In one embodiment, the disease or disorder is cancer. In another embodiment, the disease or disorder is an autoimmune or an inflammatory disease, or wherein said disease or disorder is a non-malignant abnormal growth condition, such as, cysts, for example renal cysts, thyroid cysts and thyroid masses, or hepatic cysts.

### BRIEF DESCRIPTION OF THE FIGURES

FIG. 1 are graphs showing the binding affinity of chimeric antibody 5F3 and humanized antibody SP66 to a human MUC1 expressing SKOV3 cell line using flow cytometry as described in Example 1.

FIG. 2A are graphs showing the dose dependent binding assessment of different variants (HM1 LM2, HM2 LM1, HM2 LM2, HM3 LM2, HM4 LM2, HM4 LM3 and HM5 LM2) of humanized anti-MUC1 antibody SP66 compared to chimeric 5F3 by flow cytometry on a human MUC1 expressing DA3-TM cell line.

FIG. 2B are graphs showing the dose dependent binding assessment of different variants, i.e. HM5 LM3 and HM5 LM4 of humanized anti-MUC1 antibody 5F3, compared to chimeric 5F3 by flow cytometry on a human MUC1 expressing DA3-TM cell line.

FIG. 3A is a graph showing geometric mean fluorescence plotted against different concentrations of variants of humanized anti-MUC1 5F3 as well as chimeric 5F3 analyzed by flow cytometry on a human MUC1 expressing DA3-TM cell line.

FIG. 3B is a graph showing geometric mean fluorescence plotted against different concentrations of variants of humanized anti-MUC1 5F3, i.e. HM5 LM3 and HM5 LM4 of humanized anti-MUC1 antibody 5F3, as well as chimeric 5F3 analyzed by flow cytometry on a human MUC1 expressing DA3-TM cell line.

FIGS. 4A-4D are representative flow cytometry histograms for surface expression of MUC1-SEA by chimeric IgG1 anti-MUC1-SEA mAb (5F3) (red) against the non-binding rituximab as isotype control (blue) on various human cancer cell lines. Below each histogram are the list of human cancer cell lines assessed and found to be MUC1-SEA (+ or ++ or +++ or −) as compared to isotype control.

FIGS. 5A-5E are flow cytometry histograms for surface expression of MUC1-SEA by humanized IgG1 anti-MUC1-SEA mAb (HM5, LM4) (blue) against the non-binding rituximab as isotype control (red) on human cancer cell lines viz. HT-29 (A), MCF7 (B), Calu-3 (C), SKOV3 (D) and COLO357 (E).

FIGS. 6A-6C are histograms for dose dependent binding of humanized IgG1 anti-MUC1-SEA mAb (HM5 LM4) on human cancer cell lines COLO-357 (A), SKOV-3 (B) and HT-29 (C).

FIG. 6D is a graph of concentration of humanized IgG1 anti-MUC1-SEA mAb (HM5 LM4) versus geometric mean of fluorescence plotted for MUC1-SEA expression on COLO-357, SKOV-3 and HT-29 cells.

FIG. 7 shows surface as well as cytoplasmic staining by humanized IgG1 anti-MUC1-SEA mAb (red) on MUC1 expressing COLO-357 cell line indicating the binding as well as the internalization. Hoechst is used to counter-stain nucleus (blue). Magnification was 40×.

FIG. 8 shows the chemical structure of the antibody drug conjugate HM5LM4-mc-vc-PABC-MMAE.

FIGS. 9A-9D are histograms of comparable dose dependent binding of HM5LM4-ADC (A) and humanized IgG1 anti-MUC1-SEA mAb (B) on COLO-357 cell line. Non-binding rituximab conjugated to MMAE was used as isotype ADC control (C) and rituximab was used as Isotype mAb (D).

FIG. 9E is a graph of concentration versus geometric mean of fluorescence plotted for HM5LM4-ADC, humanized IgG1 anti-MUC1-SEA mAb (HM5 LM4), isotype ADC and isotype on COLO-357.

FIGS. 10A and 10B are graphs showing a single cycle kinetic profile of humanized IgG1 anti-MUC1-SEA mAb (HM5 LM4) (10A) and HM5LM4-ADC (10B) on recombinant human MUC1-SEA on SPR.

FIGS. 11A and 11B are graphs of percentage of cell survival after contact with HM5LM4-ADC or isotype ADC (FIG. 11A) or free MMAE (FIG. 11B) on HT-29 cells.

FIGS. 12A and 12B are graphs of percentage of cell survival after contact with HM5LM4-ADC or isotype ADC (FIG. 12A) or free MMAE (FIG. 12B) on COLO-357 pancreatic carcinoma cells.

FIGS. 13A and 12B are graphs of percentage of cell survival after contact with HM5LM4-ADC or isotype ADC (FIG. 13A) or free MMAE (FIG. 13B) on BxPC3 pancreatic carcinoma cells.

FIGS. 14A and 14B are graphs of percentage of cell survival after contact with HM5LM4-ADC or isotype ADC (FIG. 14A) or free MMAE (FIG. 14B) on SKOV3 cells.

FIGS. 15A and 15B are graphs of percentage of cell survival after contact with HM5LM4-ADC or isotype ADC (FIG. 15A) or free MMAE (FIG. 15B) on T47D breast carcinoma cells.

FIG. 16A is a diagram of the wild-type MUC1-X extracellular domain (MUC1-X-FL-WT) showing the subsequences responsible for forming the loop, β-sheet, and α-helix, as described in Example 2.

FIG. 16B shows the wild type MUC1-X-FL and its mutants, MUC1-X-FL-1116 and MUC1-X-FL-1118, generated for epitope screening. The position and the amino acid residues mutated are mentioned within boxes. The downward facing arrow between glycine and serine residues indicates the cleavage site in the MUC1-SEA domain.

FIG. 17 is a bar graph showing the binding (as measured by optical density at 450 nm) of chimeric antibodies DMB5F3 (also referred to as 5F3), DMB4F4 (4F4), DMB7F3 (7F3), and DMB10F10 (10F10) as well as SP66 (the humanized form of the 5F3 antibody) and an anti-CD20 IgG antibody to MUC1-X-FL-WT (left of each individual antibody bar graph), MUC1-X-FL-1116 (middle), and MUC1-X-FL-1118 (right), as described in Example 2.

FIG. 18 is a graph showing tumor volume (mm³) over time in an ovarian cancer xenograft model after administration of vehicle (10 ml/kg, i.v., Q4D×6), HM5/LM4 (6 mg/kg, i.v., Q4D×6), HM5LM4-ADC (1.5, 3, or 6 mg/kg, i.v., Q4D×6), or 0.12 mg/kg free MMAE payload (equivalent to 6 mg/kg HM5LM4-ADC) as described in Example 4.

FIG. 19 is a graph showing tumor volume (mm³) over time in a pancreatic cancer xenograft model after administration of vehicle (10 ml/kg, i.v., Q4D×6), HM5LM4-ADC (1, 1.5, 2, or 3 mg/kg, i.v., Q4D×6), or rituximab ADC (3 mg/kg, i.v., Q4D×6) as described in Example 4.

FIG. 20 is a graph showing tumor volume (mm³) over time in a breast cancer xenograft model after administration of vehicle (10 ml/kg, i.v., Q4D×6), HM5LM4-ADC (1, 2, or 3 mg/kg, i.v., Q4D×6), rituximab ADC (3 mg/kg, i.v., Q4D×6), or free MMAE (0.06 mg/kg) as described in Example 4.

FIG. 21 is a graph showing tumor volume (mm³) over time in a head and neck cancer xenograft model after administration of vehicle (10 ml/kg, i.v., Q4D×6), HM5LM4-ADC (1, 2, or 3 mg/kg, i.v., Q4D×6), rituximab ADC (3 mg/kg, i.v., Q4D×6), or free MMAE (0.06 mg/kg) as described in Example 4.

FIG. 22 is a graph showing total flux (in photons/sec) over time in a pancreatic cancer xenograft model after administration of vehicle (10 ml/kg, i.v., Q4D×6), HM5LM4-ADC (3 mg/kg, i.v., Q4D×6), rituximab ADC (3 mg/kg, i.v., Q4D×6), or free MMAE (0.06 mg/kg) as described in Example 4.

FIG. 23 is a graph showing tumor volume (mm³) over time in a lung cancer xenograft model after administration of vehicle (10 ml/kg, i.v., Q4D×6), HM5LM4-ADC (3 mg/kg, i.v., Q4D×6), or rituximab ADC (3 mg/kg, i.v., Q4D×6) as described in Example 4.

### DETAILED DESCRIPTION OF THE INVENTION

The present invention provides sequences of monoclonal antibodies (mAbs) directed against the MUC1 SEA α-β junction (termed the SEA domain), having a robust anti-cancer activity in vivo.

The invention provides sequences of antibodies directed against the MUC1 SEA domain.

The monoclonal antibodies and immunoconjugates of the present invention are useful in the treatment of various cancers including lung, prostate, breast, ovarian, head & neck, colon, and pancreatic carcinomas. For example, the monoclonal antibodies and immunoconjugates of the present invention are useful for treating COLO-357 and BxPC3 pancreatic carcinoma cells and T47D breast carcinoma cells.

The present invention therefore provides antibodies for use in the treatment of MUC1-expressing malignancies.

Therefore, in a first of its aspects, the present invention provides a monoclonal antibody (e.g. an isolated monoclonal antibody) which binds to the MUC1 SEA domain.

The term “MUC1 SEA domain” (also referred to herein as the “MUC1 SEA module”) refers to a highly conserved domain of 110 amino acids formed by the interaction of the MUC1 α-subunit with the extracellular portion of the MUC1 β-subunit. It is therefore positioned at the MUC1 α-β junction and is a cell membrane-fixed moiety.

The SEA domain as known in the art is defined as the region located between amino acids 1039 to 1148 of the human MUC1 protein (UniprotKB-P15941 MUC1_HUMAN).

The MUC1 transmembrane glycoprotein (MUC-TM) is a heterodimer consisting of an extracellular domain containing between 20 to 125 repeats of a 20 amino acid long sequence (termed the variable number tandem repeat, VNTR), a transmembrane domain, and a short cytoplasmic tail mediating intracellular signaling. MUC1 is auto-proteolytically cleaved within the SEA module. This results in a large extracellular α subunit containing the tandem repeat array bound in a strong non-covalent interaction to a transmembrane 3 subunit containing the transmembrane and cytoplasmic domains of the molecule.

In some embodiments, the humanized antibody binds to an epitope in the MUC1 SEA domain with a binding affinity (K_{D}) less than 200 pM or 100 pM. In some instances, the humanized antibody binds to an epitope in the MUC1 SEA domain with a binding affinity (K_{D}) less than 200 pM, 90 pM, 80 pM, 70 pM, 60 pM, 50 pM, or 40 pM. In some instances, the humanized antibody binds to an epitope in the MUC1 SEA domain with a binding affinity (K_{D}) less than 30 pM. In some instances, the humanized antibody binds to an epitope in the MUC1 SEA domain with a binding affinity (K_{D}) less than 20 pM.

Specifically, the present invention provides a monoclonal antibody (e.g., an isolated monoclonal antibody) which binds to MUC1 SEA domain, wherein the antibody comprises:
(a) a heavy chain variable region comprising the amino acid sequence of any one of SEQ ID NOs. 7-11, 45, and 46; and
(b) a light chain variable region comprising the amino acid sequence of any one of SEQ ID NOs. 12-15.

In one embodiment, the humanized antibody comprises:
(a) a heavy chain variable region comprising the amino acid sequence of any one of SEQ ID NOs. 7-11; and
(b) a light chain variable region comprising the amino acid sequence of any one of SEQ ID NOs. 12-15.

In one embodiment, the humanized antibody comprises:
(a) a heavy chain variable region comprising the amino acid sequence of SEQ ID NO: 8 and a light chain variable region comprising the amino acid sequence of SEQ ID NO: 12;
(b) a heavy chain variable region comprising the amino acid sequence of SEQ ID NO: 8 and a light chain variable region comprising the amino acid sequence of SEQ ID NO: 13;
(c) a heavy chain variable region comprising the amino acid sequence of SEQ ID NO: 8 and a light chain variable region comprising the amino acid sequence of SEQ ID NO: 14;
(d) a heavy chain variable region comprising the amino acid sequence of SEQ ID NO: 9 and a light chain variable region comprising the amino acid sequence of SEQ ID NO: 13;
(e) a heavy chain variable region comprising the amino acid sequence of SEQ ID NO: 9 and a light chain variable region comprising the amino acid sequence of SEQ ID NO: 14;
(f) a heavy chain variable region comprising the amino acid sequence of SEQ ID NO: 10 and a light chain variable region comprising the amino acid sequence of SEQ ID NO: 13;
(g) a heavy chain variable region comprising the amino acid sequence of SEQ ID NO: 10 and a light chain variable region comprising the amino acid sequence of SEQ ID NO: 14;
(h) a heavy chain variable region comprising the amino acid sequence of SEQ ID NO: 11 and a light chain variable region comprising the amino acid sequence of SEQ ID NO: 13;
(i) a heavy chain variable region comprising the amino acid sequence of SEQ ID NO: 11 and a light chain variable region comprising the amino acid sequence of SEQ ID NO: 14; or
(j) a heavy chain variable region comprising the amino acid sequence of SEQ ID NO: 11 and a light chain variable region comprising the amino acid sequence of SEQ ID NO: 15.

In another embodiment, the humanized antibody comprises:
(k) a heavy chain variable region comprising the amino acid sequence of SEQ ID NO: 8 and a light chain variable region comprising the amino acid sequence of SEQ ID NO: 15;
(l) a heavy chain variable region comprising the amino acid sequence of SEQ ID NO: 9 and a light chain variable region comprising the amino acid sequence of SEQ ID NO: 12;
(m) a heavy chain variable region comprising the amino acid sequence of SEQ ID NO: 9 and a light chain variable region comprising the amino acid sequence of SEQ ID NO: 15;
(n) a heavy chain variable region comprising the amino acid sequence of SEQ ID NO: 10 and a light chain variable region comprising the amino acid sequence of SEQ ID NO: 12;
(o) a heavy chain variable region comprising the amino acid sequence of SEQ ID NO: 10 and a light chain variable region comprising the amino acid sequence of SEQ ID NO: 15;
(p) a heavy chain variable region comprising the amino acid sequence of SEQ ID NO: 11 and a light chain variable region comprising the amino acid sequence of SEQ ID NO: 12;
(q) a heavy chain variable region comprising the amino acid sequence of SEQ ID NO: 7 and a light change variable region comprising the amino acid sequence of SEQ ID NO: 12;
(r) a heavy chain variable region comprising the amino acid sequence of SEQ ID NO: 7 and a light chain variable region comprising the amino acid sequence of SEQ ID NO: 13;
(s) a heavy chain variable region comprising the amino acid sequence of SEQ ID NO: 7 and a light chain variable region comprising the amino acid sequence of SEQ ID NO: 14; or
(t) a heavy chain variable region comprising the amino acid sequence of SEQ ID NO: 7 and a light chain variable region comprising the amino acid sequence of SEQ ID NO: 15.

In one preferred embodiment, the humanized monoclonal antibody which binds to the MUC1 SEA domain comprises a heavy chain variable region comprising the amino acid sequence of SEQ ID NO: 11 and a light chain variable region comprising the amino acid sequence of SEQ ID NO: 15.

The term “antibody” refers to a polypeptide encoded by an immunoglobulin gene that specifically binds and recognizes an antigen, in the present case the MUC1 SEA domain.

The term “monoclonal antibody”, “monoclonal antibodies” or “mAb” as herein defined refers to a population of homogenous antibodies, i.e., the individual antibodies comprising the population are identical except for possibly naturally occurring rare mutations. Monoclonal antibodies are directed against a single antigenic site (epitope).

Monoclonal antibodies may be prepared and purified by any method known in the art. For example, monoclonal antibodies may be isolated from antibody fragments displayed on phage coat proteins or prepared from B cells taken from the spleen or lymph nodes of immunized animals (e.g., rabbits, rats, mice, or monkeys).

Purification of monoclonal antibodies may be performed using any method known in the art, such as hydrophobic interaction, ion-exchange and/or size-exclusion chromatography, for example, by affinity chromatography, namely, by using an affinity column to which a specific epitope (or antigen) is conjugated. Alternatively, purification of antibodies may be based on using protein A, protein G and Protein L column chromatography.

An exemplary antibody structural unit comprises a tetramer, as known in the art. Each tetramer is composed of two identical pairs of polypeptide chains, each pair having one “light chain” and one “heavy chain.” The N-terminus of each chain defines a variable region of about 100 to 110 or more amino acids primarily responsible for antigen (epitope) recognition.

Thus, the terms “heavy chain variable region” (VH) and “light chain variable region” (VL) refer to these heavy and light chains, respectively. More specifically, the variable region is subdivided into hypervariable and framework (FR) regions. Hypervariable regions have a high ratio of different amino acids in a specific position, relative to the most common amino acid in that position. Four FR regions which have more stable amino acids sequences separate the hypervariable regions. The hypervariable regions directly contact a portion of the antigen's surface. For this reason, hypervariable regions are herein referred to as “complementarity determining regions”, or “CDRs.” The CDRs are positioned both at the heavy chain of the antibody (a “heavy chain complementarity determining region”) and at the light chain of the antibody (a “light chain complementarity determining region”).

From N-terminal to C-terminal, both light and heavy chains comprise the domains FR1, CDR1, FR2, CDR2, FR3, CDR3 and FR4. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the CDR is located.

Thus, the complementarity determining regions CDRH1, CDRH2 and CDRH3 refer to the three complementarity determining regions starting from the N-terminus of the antibody's heavy chain (also referred to herein as heavy chain complementarity determining region) and the complementarity determining regions CDRL1, CDRL2 and CDRL3 refer to the three complementarity determining regions starting from the N-terminus of the antibody's light chain (also referred to herein as light chain complementarity determining region).

The present invention encompasses antigen-binding fragments of the anti-MUC1 SEA domain monoclonal antibody of the invention. As used herein the term “antigen binding fragment” relates to a fragment of the full-length antibody which retains the antibody's specificity of binding to MUC1 SEA domain. An antigen binding fragment encompasses but is not limited to Fv, single chain Fv (scFv), single chain Fv-Fc (scFv-Fc), Fab′, Fab, F(ab′)₂ and F(ab)₂. Such fragments may be produced by any method known in the art, for example by proteolytic cleavage, using enzymes such as papain (to produce Fab fragments) or pepsin (to produce F(ab′)2 fragments). In some embodiments, the antibody fragment is selected from a single-chain Fv-Fc (scFv-Fc) molecule, single chain Fv (scFv), Fv, Fab′, Fab, F(ab′)₂, and F(ab)₂. These fragments can be produced using recombinant DNA technology.

In specific embodiments, the monoclonal antibody of the invention or the antigen-binding fragment thereof binds to the MUC1 SEA domain.

In specific embodiments, the monoclonal antibodies of the invention or the antigen-binding fragment thereof is effective in reducing tumor volume in a subject. By the term “reducing” tumor volume in the context of the present invention it is meant that the monoclonal antibody of the invention or the antigen-binding fragment thereof lowers the size of the tumor as measured by any means known in the art, by at least about 10%, 20%, 30%, 40%, 50%, 60%, 70%, 80%, 90%, 95%, or about 100% as compared to the tumor size in the absence of the antibody of the invention or the antigen binding fragment thereof. In certain embodiments the term “reducing” is meant to refer to a reduction of at least about 50%, 60%, 70%, 80%, or 90%.

The term “humanized antibody” as used herein refers to an antibody that is based on the structure of a non-human species (e.g., a mouse) whose amino acid sequence has been modified to increase its similarity to antibody variants produced naturally in humans. Methods for the preparation of humanized are known in the art.

For preparing large quantities of the antibody, a transient or a stable cell line expressing the antibody can be prepared, by transfecting mammalian cells (e.g., CHO cells) with the Ig expression vector containing heavy and light chain DNA sequences of the antibody. The antibodies may then be manufactured for example in a bioreactor system. The antibodies may be purified to clinical grade using established monoclonal antibody purification methods. Clones producing high levels of anti-MUC1 SEA domain antibody may then be selected and expanded based on antibody levels in the supernatant, as tested by any method known in the art, for example, a MUC1 SEA domain-specific ELISA assay. A master cell bank developed for the specific clone may serve as the starting growing material for all clinical grade batches.

In some embodiments, the present invention provides an anti-MUC1 SEA domain monoclonal antibody (e.g., isolated monoclonal antibody) or antigen-binding fragment thereof, where the antibody or antigen-binding fragment thereof comprises a heavy chain variable region and a light chain variable region, where the heavy chain variable region is encoded by a nucleic acid sequence which is at least 70%, or 75%, or 80%, or 85%, or 90% or more identical to the nucleic acid sequence denoted by one of SEQ ID NOs: 7-11, 45, and 46 and where the light chain variable region is encoded by a nucleic acid sequence which is at least 70%, or 75%, or 80%, or 85%, or 90% or more identical to the nucleic acid sequence denoted by one of SEQ ID NOs: 12-15.

In other embodiments, the present invention provides an anti-MUC1 SEA domain monoclonal antibody (e.g., isolated monoclonal antibody) or antigen-binding fragment thereof, where the antibody or antigen-binding fragment thereof is comprised of a heavy chain variable region and a light chain variable region, where the heavy chain variable region is encoded by a nucleic acid sequence which is at least 70%, or 75%, or 80%, or 85%, or 90% or more identical to the nucleic acid sequence denoted by SEQ ID NO: 11 and where the light chain variable region is encoded by a nucleic acid sequence which is at least 70%, or 75%, or 80%, or 85%, or 90% or more identical to the nucleic acid sequence denoted by SEQ ID NO: 15.

In some embodiments, the present invention provides an anti-MUC1 SEA domain monoclonal antibody (e.g., isolated monoclonal antibody) or antigen-binding fragment thereof, wherein said antibody or antigen-binding fragment thereof comprises a heavy chain variable region comprising the amino acid sequence denoted by one of SEQ ID NOs: 7-11, 45, 46, or a variant thereof and a light chain variable region comprising the amino acid sequence denoted by one of SEQ ID NOs: 12-15, or a variant thereof.

In some embodiments, the present invention provides an anti-MUC1 SEA domain monoclonal antibody (e.g., isolated monoclonal antibody) or antigen-binding fragment thereof, wherein said antibody or antigen-binding fragment thereof comprises a heavy chain variable region comprising the amino acid sequence denoted by SEQ ID NO: 11 or a variant thereof and a light chain variable region comprising the amino acid sequence denoted by SEQ ID NO: 15, or a variant thereof.

The present invention also encompasses variants of the heavy and light chain variable regions. The variants may include mutations in the complementarity determining regions of the heavy and light chains which do not alter the activity of the antibodies herein described, or in the framework region.

By the term “variant” it is meant sequences of amino acids or nucleotides different from the sequences specifically identified herein, in which one or more amino acid residues or nucleotides are deleted, substituted, or added. It should be appreciated that by the term “added”, as used herein it is meant any addition of amino acid residues to the sequences described herein. Variants encompass various amino acid substitutions. An amino acid “substitution” is the result of replacing one amino acid with another amino acid which has similar or different structural and/or chemical properties. Amino acid substitutions may be made based on similarity in polarity, charge, solubility, hydrophobicity, hydrophilicity, and/or the amphipathic nature of the residues involved. Typically, variants encompass conservative amino acid substitutions. Conservative substitution tables providing functionally similar amino acids are known in the art. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

Conservative nucleic acid substitutions are nucleic acid substitutions resulting in conservative amino acid substitutions as defined above.

Variants in accordance with the invention also encompass non-polar to polar amino acid substitutions and vice-versa.

In some instances, the variant includes an alanine to valine mutation. In some instances, the variant includes a serine to threonine mutation. In some instances, the variant includes an alanine to isoleucine mutation. In some instances, the variant includes a leucine to isoleucine mutation. In some instances, the variant includes a valine to an arginine mutation. In some instances, the variant includes an alanine to a phenylalanine mutation. In some instances, the variant includes a methionine to an isoleucine mutation. In some instances, the variant includes a tyrosine to a lysine mutation. In some instances, the variant includes a phenylalanine to a tyrosine mutation. In some instances, the variant includes an arginine to a tyrosine mutation. In some instances, the variant includes a valine to an isoleucine mutation.

As used herein, the term “amino acid” or “amino acid residue” refers to naturally occurring and synthetic amino acids, as well as amino acid analogs and amino acid mimetics that function similarly to the naturally occurring amino acids.

Variant sequences refer to amino acids or nucleic acids sequences that may be characterized by the percentage of the identity of their amino acid or nucleotide sequences with the amino acid or nucleotide sequences described herein (for example, the amino acid or nucleotide sequences of the heavy and light chains of the antibodies herein described).

In some embodiments, variant sequences as herein defined refer to nucleic acid sequences that encode the heavy and light chain variable regions, each having a sequence of nucleotides with at least 70% or 75% of sequence identity, around 80% or 85% of sequence identity, around 90%, 91%, 92%, 93%, 94%, 95%, 96%, 97%, 98% or 99% of sequence identity when compared to the sequences of the heavy and light chain variable regions described herein.

In some other embodiments, variant sequences as herein defined refer to amino acid sequences of the heavy and light chain variable regions, each having a sequence of amino acids with at least 70% or 75% of sequence identity, around 80% or 85% of sequence identity, around 90%, 91%, 92%, 93%, 94%, 95%, 96%, 97%, 98% or 99% of sequence identity when compared to the sequences of the heavy and light chain variable regions described herein.

By the term “activity of the antibodies” it is meant the ability of the antibodies to bind MUC1 SEA domain, and preferably to mediate cell cytotoxicity either alone or as part of an immunoconjugate with a cytotoxic moiety. The activity of the antibodies can be measured in vivo or in vitro using methods known in the art.

The binding of the antibody of the invention to its target protein may be measured for example using ELISA, surface plasmon resonance (SPR), biolayer interferometry (BLI), Western blot or immunofluorescence assays (IFA).

The biological activity of the antibodies can be measured for example in an in vivo cancer model, for example as detailed in the examples below.

In another one of its aspects the present invention provides an isolated nucleic acid molecule comprising a nucleotide sequence encoding an antibody or antigen-binding fragment thereof according to the invention.

The term “nucleic acid” or “nucleic acid molecule” as herein defined refers to a polymer of nucleotides, which may be either single- or double-stranded, which is a polynucleotide such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, single-stranded (such as sense or antisense) and double-stranded polynucleotides. The term DNA used herein also encompasses cDNA, i.e., complementary or copy DNA produced from an RNA template by the action of reverse transcriptase (RNA-dependent DNA polymerase).

The invention further provides an expression vector comprising the nucleic acid molecule (e.g., isolated nucleic acid molecule) as herein defined.

“Expression vector” sometimes referred to as expression vehicle or expression construct as used herein, encompasses vectors such as plasmids, viruses, bacteriophage, integrative DNA fragments, and other vehicles, which enable the integration of DNA fragments into the genome of the host. Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. The expression vector in accordance with the invention may be competent with expression in bacterial, yeast, or mammalian host cells, to name but few.

Yet another embodiment is a host cell transfected with the nucleic acid molecule (e.g., isolated nucleic acid molecule) according to the invention or with the expression vector according to the invention.

The term “host cells” as used herein refers to cells which are susceptible to the introduction of the isolated nucleic acid molecule according to the invention or with the expression vector according to the invention. Preferably, the cells are mammalian cells, for example CHO cells or NSO cells. Transfection of the isolated nucleic acid molecule or the expression vector according to the invention to the host cell may be performed by any method known in the art.

Yet another embodiment is an immunoconjugate comprising the antibody or antigen-binding fragment thereof according to the invention and an additional cytotoxic or therapeutic agent as described herein. The term “immunoconjugate” refers to an antibody or antigen-binding fragment thereof according to the invention that is conjugated (linked or joined) to an additional agent. Immunoconjugates may be prepared by any method known in the art, for example, by cross-linking the additional agent to the antibody according to the invention or by recombinant DNA methods. The antibody may be linked to the additional agent (such as a drug, e.g., a cytotoxic agent) through a linker. The linker may be, for instance, a protease cleavable group or non-cleavable group. In one embodiment, the linker is a maleimidocaproyl-valyl-citrullinyl-p-aminobenzyloxycarbonyl (mc-val-cit-PABC) linker. Without being bound by any particular theory, the immunoconjugate binds to a tumor cell surface and gets internalized. The linker (e.g., a protease cleavable linker) thereafter is cleaved, releasing the additional agent (e.g., cytotoxic agent).

In certain embodiments, the immunoconjugate is an immunotoxin whereby the antibody or antigen-binding fragment thereof according to the invention is conjugated to a cytotoxic agent. The term “cytotoxic agent” as used herein refers to any agent that exerts a cytotoxic effect on a cell upon contact. Examples of cytotoxic agents that may be used in the immunoconjugate of the invention include, but are not limited to alkylating drugs, anthracyclines, pyrimidine derivatives, vinca alkaloids, photodynamic drugs, platinum-containing compounds, taxanes, topoisomerase inhibitors, ribosome inactivating agents (e.g., gelonin), agents that induce DNA damage (e.g., calicheamicin), tubulin inhibitors (e.g., emtansine), anti-mitotic agents (e.g., monomethyl auristatin), or bacterial toxins. The cytotoxic agents may also be radioisotopes or cytotoxic antibodies. In one embodiment, the toxic agent is Pseudomonas exotoxin, e.g., ZZ-PE38 (ZZ IgG-binding protein fused to Pseudomonas exotoxin). In another embodiment, the cytotoxic agent is monomethyl auristatin E (MMAE).

In certain embodiments, the immunoconjugate is an immunotoxin whereby the antibody or antigen-binding fragment thereof according to the invention is conjugated to a drug or payload. Suitable drugs include, but are not limited to, camptothecins (such as camptothecin, topotecan, irinotecan, silatecan, cositecan, exatecan, lurtotecan, gimatecan, rubitecan, belotecan, deruxtecan, and SN-38), topoisomerase inhibitors, maytansinoids, calicheamycins, duocarmycins, tubulysins, amatoxins, dolastatins and auristatins such as monomethyl auristatin E (MMAE) and monomethyl auristatin F (MMAF), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof. Other suitable drugs are calicheamicins, duocarymcin A (such as CC-1065, daunorubicin, mitomycin C, bleomycin, cyclocytidine, vincristine, vinblastine, methotrexate, and taxol and derivatives thereof), platinum-based antitumor agents (such as cisplatin or derivatives thereof), a tubulysin (such as tubulysin A), an amatoxin (such as α-amanitin, β-amanitin, γ-amanitin, ε-amanitin, amanullin, amanullinic acid, amaninamide, amanin, and proamanullin), a dolastatin (such as dolastatin 10 or dolastatin 15), a radioisotope, cytokines (such as interleukines, ricin, diphtheria toxin, Pseudomonas exotoxin PE38), kinase inhibitors (such as imatinib, nilotinib, dasatinib, bosutinib, ponatinib, gefitinib, erlotinib, afatinib, osimertinib, lapatinib, neratinib, sorafenib, sunitinib, pazopanib, axitinib, lenvatinib, cabozatinib, vandetanib, regorafenib, vemurafenib, dabrafenib, trametinib, cobimetinib, crizotinib, certinib, alectinib, brigatinib, lorlatinib, ibrutinib, acalibrutinib, midostaurin, ruxolitinib, idelalisib, copanlisib, palbociclib, ribociclib and abemaciclib), MEK inhibitors (such as a MEK1 and/or MEK2 inhibitors, trametinib (GSK1120212), cobimetinib or XL518, binimetinib (MEK162), selumetinib, PD-325901, CI-1040, PD035901, and TAK-733), and KSP (kinesin spindle protein) inhibitors (such as ispinesib (SB-715992), SB743921, AZ 3146, GSK923295, BAY 1217389, MPI-0479605 and ARQ 621). In one embodiment, the drug is a nucleic acid. In another embodiment, the drug is belotecan, deruxtecan, or SN-38.

One specific embodiment is an immunoconjugate comprising (a) an isolated monoclonal antibody or antigen-binding fragment thereof which binds to MUC1 SEA domain and (b) the cytotoxic agent monomethyl auristatin E (MMAE).

Another embodiment is an immunoconjugate comprising (a) an antibody comprising a heavy chain variable region comprising the amino acid sequence of SEQ ID NO: 11 and a light chain variable region comprising the amino acid sequence of SEQ ID NO: 15, (ii) a cytotoxic agent such as MMAE, and optionally (c) a linking group (such as maleimidocaproyl-Val-Cit-PABC-). In one embodiment, the immunoconjugate reduces tumor volume upon administration to a subject with cancer.

The anti MUC1 SEA domain antibody of the invention may be administered in combination with at least one additional therapeutic agent.

The term “additional therapeutic agent” used herein refers to any agent that may be used for treating a disease or disorder, e.g., cancer.

In certain embodiments the additional therapeutic agent is an additional antibody. The term “additional antibody” as herein defined refers to antibodies of the invention (namely to the combined use of at least two antibodies of the invention) as well as to an antibody, which is not the antibody according to the invention, which may be used in combination with the antibody of the invention for treating a disease or disorder, e.g., cancer. Such other antibodies include but are not limited to anti-CD22 antibodies, anti-CD30 antibodies, anti-HER2 receptor antibodies, anti-VEGF antibodies, anti-EGFR antibodies, anti-tumor associated antigen (TAA) antibodies, and anti-check point inhibitors.

The additional therapeutic agent may also be a chemotherapeutic agent, or an anti-inflammatory agent.

Yet another embodiment is a pharmaceutical composition comprising as an active ingredient at least one anti-MUC1 SEA antibody (e.g., isolated anti-MUC1 SEA antibody) of the invention, or antigen-binding fragment thereof or the immunoconjugate as herein defined and a pharmaceutically acceptable carrier, excipient, or diluent.

In a specific embodiment, the pharmaceutical composition is for use in the treatment of a disease or disorder associated with over-expression of MUC1.

The term “a disease or disorder associated with over-expression of MUC1” is used herein at its broadest sense and refers to any disease which is characterized by aberrant expression of MUC1. In a specific embodiment, the disease or disorder associated with over-expression of MUC1 is cancer. Examples include, but are not limited to, lung carcinoma, prostate carcinoma, breast carcinoma, ovarian carcinoma, colon carcinoma, small intestinal carcinoma, pancreatic carcinoma, gastric carcinoma, liver cancer, multiple myeloma, or acute myelogenous leukemia. In one embodiment, the disease or disorder is an advanced and/or metastatic solid tumor. In another embodiment, the disease or disorder is breast carcinoma (e.g., ER+ breast carcinoma). In yet another embodiment, the disease or disorder is non-small cell lung cancer. In yet another embodiment, the disease or disorder is epithelial ovarian carcinoma.

In other embodiments, the disease or disorder associated with over-expression of MUC1 is an autoimmune or inflammatory disease. Non-limiting examples of an autoimmune or inflammatory disease include rheumatoid arthritis, psoriatic arthritis, systemic lupus erythematosus, amyloidosis, and autoimmune pancreatitis.

In other embodiments, the disease or disorder is a non-malignant abnormal growth condition, such as, cysts, for example clinically significant renal cysts, large, non-functional thyroid cysts and thyroid masses, or hepatic cysts.

The “pharmaceutical composition” of the invention may comprise the antibody or any antigen-binding fragment thereof as herein defined and a buffering agent, an agent which adjusts the osmolarity of the composition and optionally, one or more pharmaceutically acceptable carriers, excipients and/or diluents as known in the art.

As used herein the term “pharmaceutically acceptable carrier, excipient or diluent” includes any solvent, dispersion medium, coating, antibacterial and antifungal agent, as known in the art. The carrier can be a solvent or dispersion medium containing, for example, water, ethanol, polyol (for example, glycerol, propylene glycol, and liquid polyethylene glycol), suitable mixtures thereof, and vegetable oils. Each carrier should be both pharmaceutically and physiologically acceptable in the sense of being compatible with the other ingredients and not injurious to the subject. Except as any conventional media or agent is incompatible with the active ingredient, its use in the therapeutic composition is contemplated. For instance, for a pharmaceutical composition intended for intravenous administration, the pharmaceutically acceptable carrier may be a 0.9% sodium chloride solution for injection.

In other embodiments, the pharmaceutical composition according to the invention further comprises an additional therapeutic agent. Non-limiting examples of additional therapeutic agents include anti-MUC1 antibodies, anti-CD22 antibodies, anti-CD30 antibodies, anti-HER2 receptor antibodies, anti-VEGF antibodies, anti-EGFR antibodies, anti-TAA antibodies and checkpoint inhibitors.

Yet another embodiment is a method of treatment or amelioration of a disease or disorder associated with over-expression of MUC1 (e.g. cancer) comprising administering to a subject in need thereof a therapeutically effective amount of the monoclonal antibody (e.g., isolated monoclonal antibody) or antigen-binding fragment thereof of the invention, or an immunoconjugate comprising the antibody or antigen-binding fragment thereof of the invention or a pharmaceutical composition comprising the isolated monoclonal antibody or antigen-binding fragment thereof or the immunoconjugate of the invention.

The terms “subject” or “patient” are used interchangeably and refer to a subject that may benefit from the present invention such as a mammal (e.g., canine, feline, ovine, porcine, equine, bovine, or human). In one specific embodiment, the subject or patient is human. Diagnosis of a disease or disorder associated with over-expression of MUC1 may be performed by a skilled physician by methods known in the art.

The term “subject in need thereof” in the context of the present invention inter alia refers to mammals, particularly human subjects suffering from a disease or disorder associated with over-expression of MUC1, as defined herein.

It is to be understood that the terms “treat”, “treating”, “treatment” or forms thereof, as used herein, mean reducing, preventing, curing, reversing, ameliorating, attenuating, alleviating, minimizing, suppressing, or halting the deleterious effects of a disease or a condition or delaying the onset of one or more clinical indications of a disease or disorder associated with over-expression of MUC1 (e.g., cancer), as defined herein. In some embodiments the methods according to the invention are wherein said methods further comprise administering to a subject in need thereof an additional therapeutic agent as herein defined.

Administration according to the present invention may be performed by any of the following routes: oral administration, intravenous, intramuscular, intraperitoneal, intrathecal, or subcutaneous injection; intra-rectal administration; intranasal administration, ocular administration, or topical administration.

In specific embodiments administration according to the present invention is performed intravenously.

The antibodies or antibody fragments as herein defined, any pharmaceutical compositions comprising the same or any conjugates comprising them may be administered to a subject in a single dose or in multiple doses.

A “therapeutically effective amount of the isolated monoclonal antibody or any antigen-binding fragment thereof according to the invention, or the pharmaceutical composition according to the invention for purposes herein defined is determined by such considerations as are known in the art to cure, arrest or at least alleviate or ameliorate the medical condition. For any preparation used in the methods of the invention, the dosage or the therapeutically effective amount can be estimated initially from in vitro cell culture assays or based on suitable animal models.

In some embodiments the therapeutically effective amount in accordance with the invention is in the range of 10 pg/kg to about 50 mg/kg.

In other embodiments the therapeutically effective amount in accordance with the invention is in the range of 0.1 mg/kg to 40 mg/kg, 1 mg/kg to 10 mg/kg, or 5 mg/kg to 10 mg/kg.

Specific exemplary doses include, but are not limited to 0.25 mg/kg, or 0.75 mg/kg, or 2.5 mg/kg, or 5 mg/kg, or 10 mg/kg given as a daily dose, or once every three days, or once a week or once in three weeks according to the physician's discretion. In one embodiment, the doses are given intravenously.

The present invention further provides the anti MUC1 SEA antibody (e.g., the isolated anti MUC1 SEA antibody) or any antigen-binding fragment thereof according to the invention, or the immunoconjugate according to the invention, or the pharmaceutical composition according to the invention for use in a method of treatment or amelioration of a disease or disorder associated with over-expression of MUC1 (e.g., cancer) as defined herein.

Still further the present invention provides use of the monoclonal antibody (e.g., isolated monoclonal antibody) or antigen-binding fragment thereof, the immunoconjugate, or the pharmaceutical composition of the invention in the preparation of a medicament for the treatment or amelioration of a disease or disorder associated with over-expression of MUC1 (e.g., cancer), as defined herein.

It is appreciated that the term “purified’ or “isolated” refers to molecules, such as amino acid or nucleic acid sequences, peptides, polypeptides, or antibodies that are removed from their natural environment, isolated, or separated. An “isolated antibody” is therefore a purified antibody. As used herein, the term “purified” or “to purify” also refers to the removal of contaminants from a sample.

Yet another embodiment is a method of diagnosing a disease or disorder (e.g., cancer) in a biopsy obtained from a subject, said method comprising:
(a) contacting said biopsy with at least one isolated monoclonal antibody or antigen-binding fragment thereof of the invention; and
(b) detecting said isolated monoclonal antibody or any antigen-binding fragment thereof;
   wherein detection of cells over-expressing MUC1 SEA in the biopsy serves as an indication of said disease or disorder (e.g., cancer).

Assessment of the ability of the isolated antibodies of the present invention to detect MUC1-SEA expression may be performed by any method known in the art, for example by immunohistochemistry or flow cytometry. Immunohistochemistry may be performed on formaldehyde fixed sections from Fresh Frozen (FF) tissues as well as on Paraffin Embedded and Formaldehyde Fixed (PEFF) tissues. The antibody is capable of recognizing MUC1-expressing cells using flow cytometry. In various embodiments the isolated antibodies in accordance with the present invention may be labeled according to any methods known in the art. In other embodiments detection may be based on identifying said antibodies using secondary antibodies.

The term “biopsy” is used herein in its broadest sense and refers to any biopsy taken from a subject as herein defined in which cells overexpressing MUC1 SEA may be detected. Biopsies may be obtained from mammals (including humans) and encompass both fluid samples comprising cells, and tissue samples. In some embodiments the fluid sample is blood, plasma, serum, lymph fluid or urine. In some embodiments the biopsy is a tissue sample suspected of containing cancerous cells.

In another aspect, the present invention provides a method of imaging a disease or disorder, said method comprising:
(a) introducing at least one isolated anti-MUC1 SEA monoclonal antibody or antigen-binding fragment thereof of the invention into a subject, wherein said antibody or antigen-binding fragment thereof are detectably labelled with a radioisotope, or with a visualizable agent (i.e., an agent that can be visualized, for example by scanning); and
(b) visualizing said detectably labelled isolated anti-MUC1 SEA monoclonal antibody or any antigen-binding fragment thereof,
   wherein detection of cells and/or tissues labelled with said isotope or with said visualizable agent indicates the presence, and/or the localization and/or the extent and/or the presence of metastases of said disease or disorder in said subject.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. Disclosed and described, it is to be understood that this invention is not limited to the specific examples, methods steps, and compositions disclosed herein as such methods steps and compositions may vary somewhat. It is also to be understood that the terminology used herein is used for the purpose of describing specific embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. The term comprising will also be understood to include “consisting of” and “consisting essentially of.”

### EXAMPLES

### Example 1

### Humanized Anti-MUC1 Antibody

The chimeric antibody 5F3 (also referred to as chimeric DMB5F3) described in International Publication No. WO 2021/186427, was humanized as follows. A human framework was used as a template to generate a humanized version of anti-MUC1 α/β mAb, 5F3. The sequences of the variable region of the heavy and light chains of chimeric antibody 5F3 are provided in SEQ ID NOs: 42 and 43, respectively.

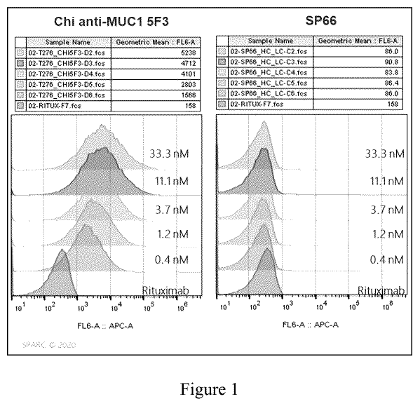

The CDR residues from mouse anti-MUC1 antibody (m5F3) VH- (SEQ ID NO: 1) and VL- (SEQ ID NO: 2) domains were identified according to the three antibody numbering systems and grafted onto the human framework region sequences VH- (SEQ ID NO: 3) and VL- (SEQ ID NO: 4) domains respectively. CDRs from 5F3 were chemically synthesized and subsequently cloned into mammalian expression vectors. The expression vectors with humanized 5F3 heavy chain and light chain were named as SP66-HC and SP66-LC respectively. The VH domain of SP66 is SEQ ID NO: 5, and the VL domain of SP66 is SEQ ID NO: 6. The antibody expressed with SP66-HC and SP66-LC was named as SP66. SP66 was transiently expressed in CHO cells and purified by Protein-A affinity chromatography and evaluated for its binding to the human MUC1 SEA domain using flow cytometry on the human MUC1-expressing SKOV3 cell line (FIG. 1). The results of this binding study clearly showed that despite having the same CDRs, the SP66 IgG product was unable to bind to human MUC1 SEA domain using this technique.

Furthermore, SP66 was tested against human cancer cell lines COLO357, SKOV-3, and HT29. An anti-CD20 IgG antibody was used as a control. No growth inhibition was observed in any of the cell lines after treatment with SP66.

Some of the Vernier-zone residues of mouse 5F3 mAb were back mutated into SP66. A number of constructs were generated with different combinations of mutations introduced into the framework regions of SP66-VH and -VL domains. These constructs were named as HM1 (SEQ ID NO: 7), HM2 (SEQ ID NO: 8), HM3 (SEQ ID NO: 9), HM4 (SEQ ID NO: 10), HM5 (SEQ ID NO: 11), HM6 (SEQ ID NO: 45), and HM7 (SEQ ID NO: 46) for the variants of SP66-VH domain and LM1 (SEQ ID NO: 12), LM2 (SEQ ID NO: 13), LM3 (SEQ ID NO: 14), and LM4 (SEQ ID NO: 15) for the variants of SP66-VL domain (Table 1).

TABLE 1
List of mutations introduced in VH & VL domains of humanized
anti-MUC1 mAb (SP66) to restore the binding affinity

| Clones of SP66 | Mutations |
|---|---|
| Heavy chain (HM1) | A24V, S25T |
| Heavy chain (HM2) | A24V, S25T, A67I, L69I, V71R |
| Heavy chain (HM3) | A24V, S25T, V71R |
| Heavy chain (HM4) | A24V, S25T, V72R, A78F |
| Heavy chain (HM5) | A24V, S25T, A671, L69I, V71R, A78F |
| Heavy chain (HM6) | V71R, A78F |
| Heavy chain (HM7) | A67I, L69I, V71R, A78F |
| Light chain (LM1) | M48I, Y49K |
| Light chain (LM2) | M48I, Y49K, F36Y |
| Light chain (LM3) | M48I, Y49K, F36Y, R50Y |
| Light chain (LM4) | M48I, Y49K, F36Y, R50Y, V106I |

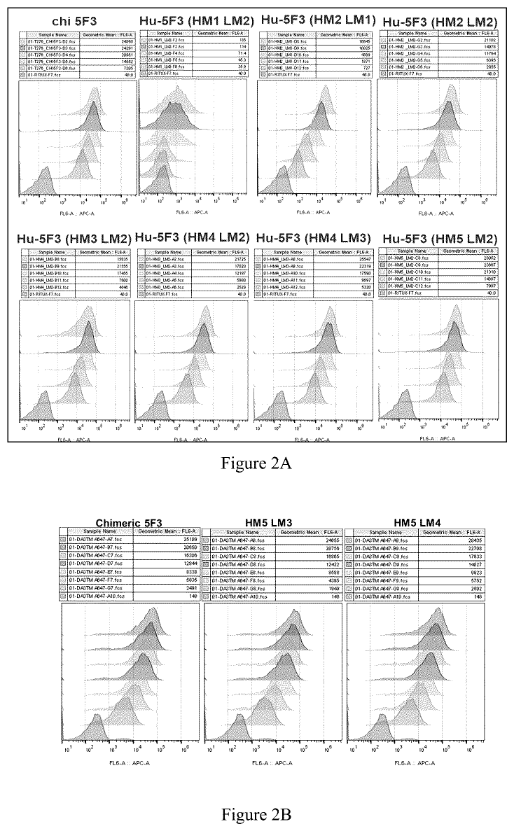

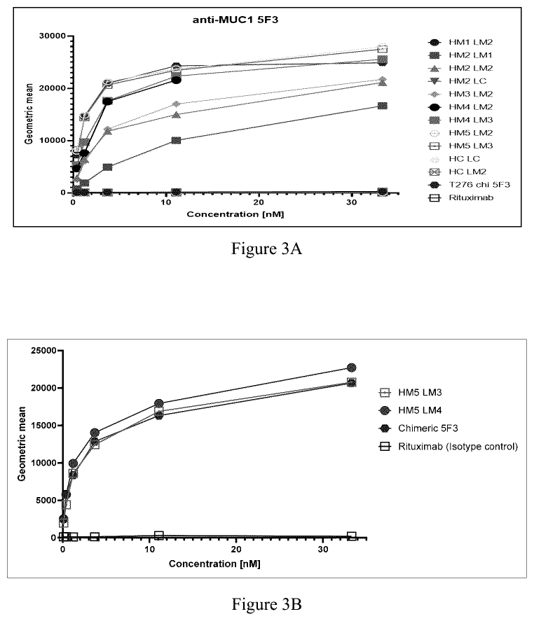

These variants were expressed in CHO cells, purified, and evaluated for their binding to MUC1 via surface plasmon resonance (SPR) or flow cytometry. The binding data showed improve in binding of various variants of SP66 to MUC1 compared to SP66 (Tables 2 and 3 below, and FIGS. 2A, 2B, 3A, and 3B). The final version of the humanized antibody was named as HM5/LM4. The binding data showed that HM5/LM4 have a similar binding affinity to MUC1 compared to that of parent 5F3 antibody.

TABLE 2
Binding affinity of different variants of humanized
anti-MUC1 5F3 mAb determined by SPR

| Sample Name | ka (1/Ms) | kd (1/s) | KD (M) |
|---|---|---|---|
| chi 5F3 | 4.39E+06 | 1.88E−04 | 4.27E−11 |
| HM2 LM2 | 1.77E+06 | 2.55E−04 | 1.44E−10 |
| HM2 LM3 | 3.14E+06 | 1.88E−04 | 5.98E−11 |
| HM3 LM2 | 2.65E+06 | 4.92E−04 | 1.86E−10 |
| HM3 LM3 | 4.47E+07 | 3.47E−03 | 7.75E−11 |
| HM4 LM2 | 4.08E+06 | 2.61E−04 | 6.39E−11 |
| HM4 LM3 | 2.88E+06 | 2.38E−04 | 8.26E−11 |
| HM5 LM2 | 4.80E+06 | 2.13E−04 | 4.44E−11 |
| HM5 LM3 | 3.97E+06 | 1.98E−04 | 5.00E−11 |
| HM5 LM4 | 5.02E+06 | 1.50E−04 | 2.98E−11 |

TABLE 3
Binding assessment of different variants of humanized
and chi anti-MUC1 antibody by flow cytometry
on MUC1 positive DA3-TM cell line

| Sample Name | KD (nM) |
|---|---|
| Chimeric 5F3 | 1.0 ± 0.1 |
| HM2 LM1 | 14.5 ± 0.8 |
| HM2 LM2 | 3.2 ± 0.2 |
| HM3 LM2 | 3.7 ± 0.1 |
| HM4 LM2 | 2.1 ± 0.3 |
| HM4 LM3 | 1.5 ± 0.6 |
| HM5 LM2 | 1.4 ± 0.6 |
| HMS LM3 | 0.9 ± 0.2 |
| HM5 LM4 | 1.0 |

### Methods

Cloning of VH and VL Domains into Mammalian Expression Vectors

The gene fragments (SEQ ID NO: 5 and SEQ ID NO: 6) were chemically synthesized. The DNA sequence of the VH-domain (SEQ ID NO: 5) was amplified by PCR using primers SP190-FP and SP191-RP (SEQ ID NOs: 16 and 17, Table A) Vent DNA Polymerase (NEB), according to manufacturer's protocol. The resulting PCR product was digested with BSSHII and NheI restriction enzymes and inserted into the pMAZIgH vector that was cut with same enzymes. Similarly, VL-domain (SEQ ID NO: 6) was PCR amplified using SP192-FP and SP193-RP primers (SEQ ID NOs: 18 and 19, respectively) and inserted into the pMAZIgL vector after digestion with BSSHII and BSiWI restriction enzymes. All the constructs were confirmed by DNA sequencing.

### Construction of Humanized Versions of Anti-MUC1 Antibody, 5F3

The Vernier zone residues in the VH and VL domains of SP66 antibody were mutated to the corresponding residues present in mouse anti human MUC1 antibody, m5F3. The amino acid mutations were carried out using the Q5 Site-Directed Mutagenesis Kit (NEB) according to the manufacturer's instructions. The sequences of primers used for each of these mutations are provided at SEQ ID NOs:16-36 and SEQ ID NO:44. in Table A. The templates and primer used to generate different versions of humanized antiMUC1, 5F3 are given in Table 4. The amino acid positions mutated are according to the Kabat numbering system.

TABLE 4
Templates and the list of primers used to generate different versions
of VH & VL domains of humanized anti-MUC1 mAb
(SP66) to restore the binding affinity

| | | | Primers |
|---|---|---|---|
| | | | used |
| Clones of SP66 | Mutations | Template used in PCR | in PCR |
| Heavy chain (HM1) | A24V, S25T | SP66-Heavy chain | SP223-FP |
| | | | SP224-RP |
| Heavy chain (HM2) | A24V, S25T, | SP66 heavy chain, HM1 | SP227-FP |
| | A67I, L69I, | | |
| | V71R | | SP228-RP |
| Heavy chain (HM3) | A24V, S25T, | SP66 heavy chain, HM1 | SP229-FP |
| | V71R | | SP230-RP |
| Heavy chain (HM4) | A24V, S25T, | SP66 heavy chain, HM1 | SP231-FP |
| | V71R, A78F | | SP233-RP |
| Heavy chain (HM5) | A24V, S25T, | SP66 heavy chain, HM2 | SP231-FP |
| | A67I, L69I, | | |
| | V71R, A78F | | SP270-RP |
| Heavy chain (HM6) | V71R, A78F | SP66 heavy chain, HM4 | SP291-FP |
| | | | SP224-RP |
| Heavy chain (HM7) | A67I, L69I, | SP66 heavy chain, HM5 | SP291-FP |
| | V71R, A78F | | SP224-RP |
| Light chain (LM1) | M48I, Y49K | SP66 light chain, | SP270-RP |
| | | | SP240-RP |
| Light chain (LM2) | M48I, Y49K, | SP66 light chain, LM1 | SP237-FP |
| | F36Y | | SP238-RP |
| Light chain (LM3) | M48I, Y49K, | SP66 light chain, LM1 | SP268-FP |
| | F36Y, R50Y | | SP269-RP |
| Light chain (LM4) | M48I, Y49K, | SP66 light chain, LM3 | SP332-FP |
| | F36Y, R50Y, | | SP333-RP |
| | V106I | | |

### Example 2

### Human MUC1 SEA Epitope Mapping

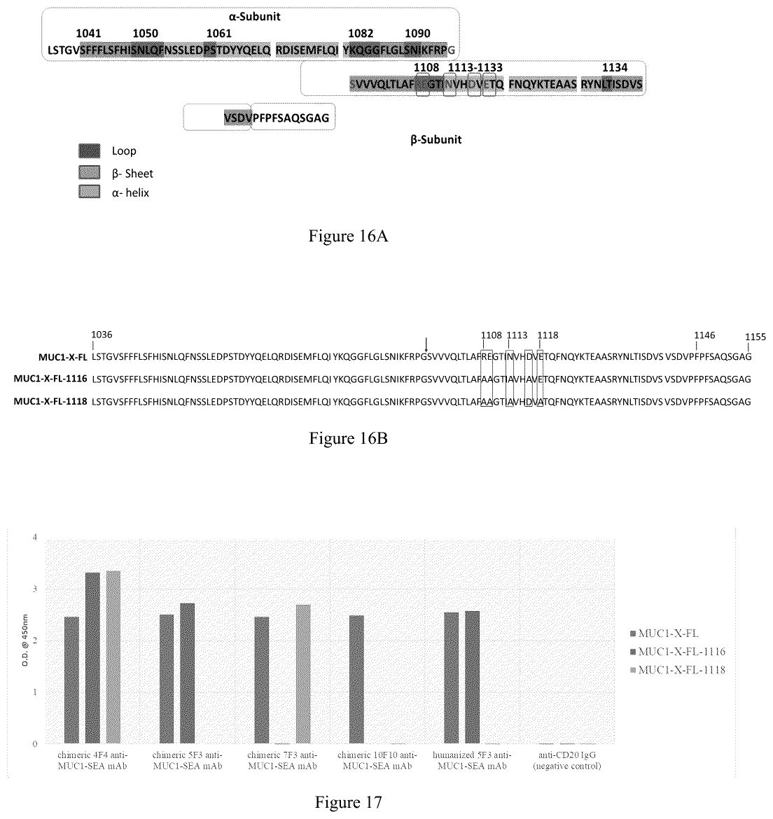

The sequences of wild-type MUC1-X extracellular domain (MUC1-X-FL-WT) (a subsequence of MUC1, SEQ ID NO: 41) was used to generate various mutants on SEA domain. Mutations were carried out using site directed mutagenesis approach at various site of MUC1 SEA. Mutants referred to as MUC1-X-FL-1 116 (mutations of R1108A, E1109A, N1113A, D1116A) and MUC1-X-FL-1118 (mutations of R1108A, E1109A, N1113A, E1118A), as shown in FIG. 16B, were generated and cloned in a pET30a (+) expression vector. These recombinant proteins were expressed in E. coli and isolated using Ni-NTA columns. ELISA method was used to determine the binding of 5F3 antibody to these mutants.

FIG. 16A is a pictorial diagram of the wild-type MUC1-X extracellular domain (MUC1-X-FL-WT, a subsequence of human MUC1, SEQ ID NO: 41).

Chimeric antibodies DMB5F3 (also referred to as 5F3), DMB4F4 (4F4), DMB7F3 (7F3), and DMB10F10 (10F10) as well as SP66 and HM5 LM4 (the humanized form of the 5F3 antibody) and an anti-CD20 IgG antibody were tested for binding to MUC1-X-FL-WT, MUC1-X-FL-1116, and MUC1-X-FL-1118. Each of the chimeric antibodies is described in International Publication No. WO 2021/186427. The results are shown in FIG. 17. Binding of 5F3 antibody and its humanized version, HM5 LM4, to MUC1 is lost when there is a mutation at position 1118 of MUC1 (MUC1-X-FL-1118), but is maintained when there is a mutation at position 1116 (MUC1-X-FL-1116). In contrast, binding affinity of the 7F3 antibody to MUC1 is lost when there is a mutation at position 1116 (MUC1-X-FL-1116), but is maintained when there is a mutation at position 1118 (MUC1-X-FL-1118). Binding affinity of the 10F10 antibody is compromised when there is a mutation at position 1116 or 1118.

### Example 3

### Humanized Anti-MUC1 Antibody Drug Conjugate

Expression of humanized anti-MUC1 SEA antibody in CHO Cells: Plastic non-adherent CHO-S cells adapted for growth under serum-free conditions were used for high level transient expression of humanized anti-MUC1 SEA antibody protein. CHO-S cells were transfected with 0.8 μg/mL to 1.5 μg/mL of plasmid DNA encoding humanized anti-MUC1 SEA antibody sequence using the Expi-CHO Expression System Kit according to the manufacturer's instructions (Thermo Fisher (Waltham, MA), Cat. A29133). 24 hours post-transfection, the transfected cell cultures were diluted to 16% volume/volume using Expi-CHO feed and enhancer (Thermo Fisher, Cat. A29129) and allowed to grow for additional 14 days in non-baffled flasks in a humidified 5% CO₂ shaker incubator at 32° C. at 120 rpm. After 14 days, cell-free supernatants were harvested and the antibody was purified using an appropriate method.

Purification: CHO culture-broth containing secretory mAb was harvested at the end of the production stage and was subjected to centrifugation followed by 0.2 μm filtration to remove the cells and any debris and insoluble materials. Then EDTA was added to this cell-free culture supernatant at a final concentration of 2 mM. This culture supernatant was then purified by Protein A column chromatography using an AKTA Avant (GE Healthcare Ltd., Little Chalfont, Buckinghamshire, UK) chromatography system. Briefly, Protein A pre-packed column(s) (5 mL or its multiples), MabSelect Sure™ (GE Healthcare Ltd., Little Chalfont, Buckinghamshire, UK) was equilibrated by passing 3-5 bed volumes of equilibration buffer containing 25 mM Tris-Cl pH 7.0, 100 mM NaCl. Reconditioned culture supernatant was loaded onto equilibrated protein A affinity column at a flow rate to achieve optimum residence time (RT of 2-3 min) depending on the antibody-titer considering ≤35 mg/mL dynamic binding capacity of the resin (as per manufacturer's instructions), followed by washing of the column 3-5 bed volumes of equilibration buffer to remove any unbound proteins. Further, the column was washed with 3-5 bed volume of a high ionic strength buffer containing 25 mM Tris-Cl pH 7.0, 1M NaCl to remove any non-specifically bound proteins or media components. The column was then washed by passing 3-5 bed volumes of a low-pH buffer containing 50 mM sodium acetate pH 6.0. Elution of bound protein was then performed at an acidic pH by passing 5-7 bed volumes of the elution buffer containing 25 mM sodium acetate pH 3.5, 100 mM NaCl. Eluted protein fraction was incubated at room temperature for 45-60 minutes for low-pH viral inactivation and then pH was adjusted to 5.5 with 2M Tris solution. Finally, the protein A column was cleaned by passing 0.1 N NaOH solution followed by Milli-Q water and stored by passing 20% ethanol at 2-8° C., as per the manufacturer's instructions. Protein A-purified protein samples were formulated by adding sucrose (250 mM final concentration) and polysorbate 20 (0.01% v/v final concentration) followed by 0.2 μm filtration. Purified protein was stored at 2-8° C. for short-term or −20° C. for long-term storage.

Antibody conjugation of monomethyl auristatin E (MMAE) for ADC generation: The humanized IgG1 anti-MUC1-SEA mAb HM5-LM4 was buffer exchanged into conjugation buffer (20 mM Sodium Phosphate, 2 mM EDTA, pH 7.4) for partial reduction with 2.2 molar excess of TCEP at 37° C. for ˜120 minutes. After reduction, it was buffer exchanged through either 30 kDa centrifugal ultrafiltration or by Tangential Flow Filtration (TFF) or Sephadex G25 resin (a gel filtration medium) with conjugation buffer. The thiol generated were quantified by Ellman's reagent, 5,5′-dithiobis(2-nitrobenzoic acid) [DTNB]. A 10 fold molar excess of maleimidocaproyl-Val-Cit-PABC-MMAE (vcMMAE) was added into the reduced antibody. The conjugation reaction was carried out in the presence of ˜10% DMF and 100 mM sodium caprylate at 22° C.±3° C. for 60 minutes. After the conjugation, 20 fold molar excess of cysteine to vcMMAE was added to quench the unreacted vcMMAE. The conjugate was buffer exchanged through either 30 kDa centrifugal ultrafiltration or by TFF or Sephadex G25 resin (a gel filtration medium) with HIC (hydrophobic interaction chromatography) binding buffer and further purified through preparative HIC chromatography to enrich drug-to-antibody ratio (DAR) 2 & 4 species. HIC purified ADC was concentrated and buffer exchanged through 30 kDa MWCO centrifugal ultrafiltration or TFF into succinate buffer with 9.0% (w/v) sucrose and 0.04% Tween 80, pH 5.5 and stored either at 4° C. or −20° C. for further uses. The ADC generated is referred to as HM5LM4-ADC.

Screening of surface MUC1 expression on human cancer cells using flow cytometer: Human cancer cell lines (including A253, COLO357, HT29, SKOV3, etc.) were harvested enzymatically using 0.25% Trypsin and 0.2% EDTA in Dulbecco's Phosphate Buffered Saline (D-PBS), washed with excess cell culture media and re-suspended at a cell density of 1-2×10⁶/mL in the cell-line specific cell culture media. The anti-MUC1-SEA mAb (chimeric or humanized IgG) or the reference IgG antibody were added to cells at a specific concentration. The cells were maintained at 4° C. for 1 hour and washed with the excess cell culture media. These cells were further stained with goat anti-human IgG, Fcγ, conjugated with Alexa Fluor 647 (Jackson Immuno Research, West Grove, PA, USA) for 1 hour at 4° C. The concentration for the goat anti-human IgG, Fcγ was as per the manufacturer's instructions. Cells were further washed with the excess cell culture media and re-suspended in 100 μl of cell culture media. Cell-associated fluorescence was detected and recorded using flow cytometry (Beckman Coulter, Cytoflex). One log shift towards right of the peak of auto-fluorescence of isotype control cells was considered as one log shift (or MUC1+) and the subsequent shifts as two (or MUC1++), three (or MUC1+++) and so forth.

Dose dependent binding of surface MUC1 expression on human cancer cells using flow cytometer: Human cancer cell lines (COLO357, HT29 and SKOV3) or mouse cancer cell line expressing human MUC1 (DA3-TM) were harvested enzymatically using 0.25% Trypsin and 0.2% EDTA in Dulbecco's Phosphate Buffered Saline (D-PBS), washed with excess cell culture media and re-suspended at a cell density of 1-2×10⁶/mL in the cell-line specific cell culture media. The anti-MUC1-SEA mAb (chimeric or humanized IgG)/ADC or the reference IgG antibody/ADC were added to cells at a concentration range of 0-50 nM. The cells were maintained at 4° C. for 1 hour and washed with the excess cell culture media. These cells were further stained with goat anti-human IgG, Fcγ, conjugated with Alexa Fluor 647 (Jackson Immuno Research, West Grove, PA, USA) for 1 hour at 4° C. The concentration for goat anti-human IgG, Fcγ was as per the manufacturer's instructions. Cells were further washed with the excess cell culture media and re-suspended in 100 μl of cell culture media. Cell-associated fluorescence was detected and recorded using flow cytometry (Beckman Coulter, Cytoflex). The mean intensity of fluorescence was obtained from FlowJo software and graph was plotted in Graphpad prism of concentration versus mean intensity of fluorescence.

Internalization of cell bound anti-MUC1-SEA antibody using flow cytometry: The procedure in E. G. Kim et. al., Biomolecules 2020, 10, 955; doi:10.3390/biom10060955, was slightly modified where the percentage of internalization of a cell surface-bound antibody inside the cells was determined by comparing the net decrease in geometric mean of fluorescence (MFI) of secondary antibody normalized to primary antibody signals incubated at 37° C. at 10 μg/ml concentration to the corresponding control MFI of secondary antibody normalized to primary antibody signals maintained at 0 min. The relative MFI of the sample at each time point x (t=x is 0, 0.5, 1, 2 and 4 hours) to the percentage of the control was calculated as follows:
Normalized MFI (surface/total)=MFI from secondary antibody/MFI from primary antibody
Relative MFI (% of control)=(MFI surface/totalₜ₌ₓ/MFI surface/totalₜ₌₀)×100, where
MFIₜ₌₀ is the MFI of the control at 0 time
MFIₜ₌ₓ is the MFI of the matching sample at 37° C. at the indicated time point x.
The percentage of antibody internalization at each time point x was then calculated as

$$Internalization ( \% ) = 100 \% - Relative MFI ( \% of control )$$

Internalization assessment of anti-MUC1-SEA antibody using microscopy: 10,000 Colo357 cells seeded in 8-well chamber slides were treated with 10 μg/ml of Alexa647 labelled antibody or Alexa647 labelled isotype control and incubated for 24 hours at 37° C. in a humidified incubator set at 5% CO₂. Post 24 hours treatment, cells were washed with complete media and then fixed with 2% paraformaldehyde at room temperature (RT) for 10-15 minutes (min). Following fixation, cells were washed, counter stained for nucleus using Hoechst dye for 5 min and mounted with coverslip using fluoromount-G. Cells were imaged using fluorescence microscope at 40×objective and images were processed in ImageJ software. Alexa647 signals were shown in red whereas nucleus counterstain was shown as blue.

Binding strength assessment using Surface Plasmon Resonance (SPR): MUC1 antigen (Acrobiosystems, Cat: MU1-H52H9) was captured on the anti-His immobilized on CM5 chip at 5 μl/min flow rate for 60 s. Post this concentration range of 0-50 nM of antibody or ADC is passed over MUC1 antigen at a flow rate of 30 μl/min flow rate for 120 s, keeping dissociation time of 1800 s at 25°. The dose response generated is evaluated on BIAevaluation software for the calculation of affinity of antibody to antigen as measured by dissociation constant (KD).

### Growth Inhibition of Human Cancer Cell-Lines Using ADC of MMAE

Human cancer cell lines (such as Colo357, HT29, SKOV3, BxPC3 and T47D) were seeded at 5000 cells/well in a 96-well plate flat bottom clear plate and allowed to adhere overnight. Next day, antibody/ADCs were added to these cells at the mentioned concentration or at a mentioned concentration range. Post 5-7 days of treatment, 20 μl of presto blue dye was added to wells and absorbance was recorded at 570 nm (600 nm reference wavelength). Percent survival was calculated against wells containing untreated cells in complete media and IC50 was calculated in Graphpad Prism.

### Results

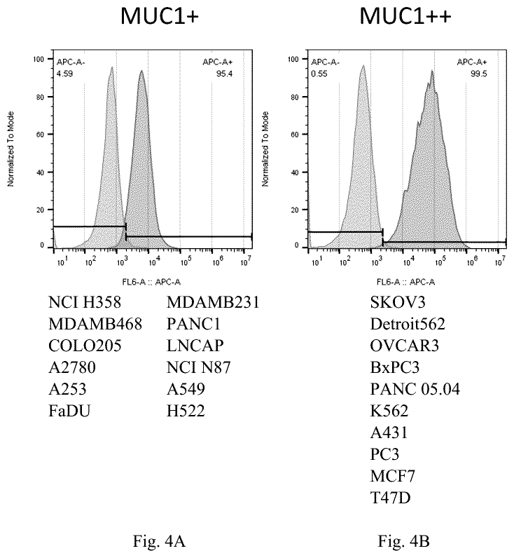

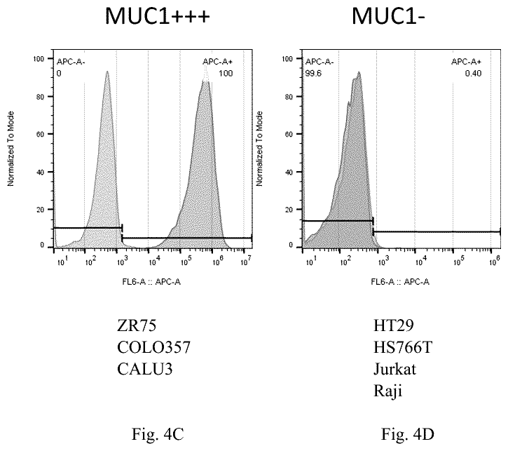

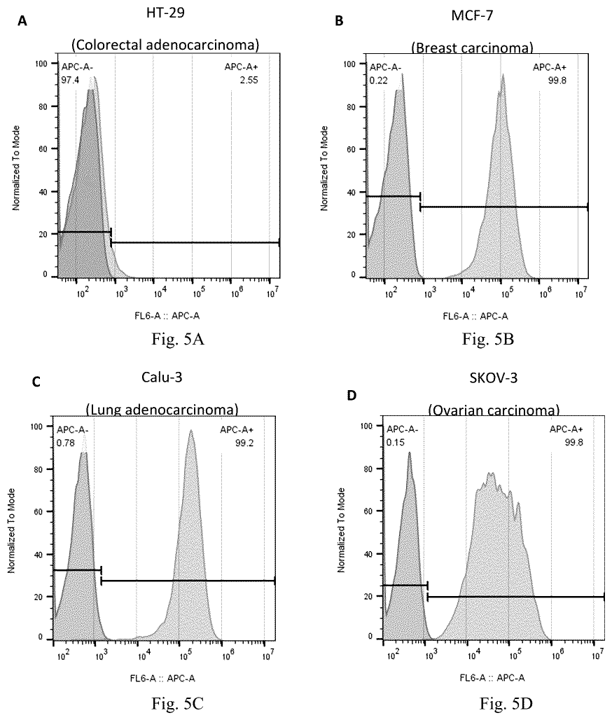

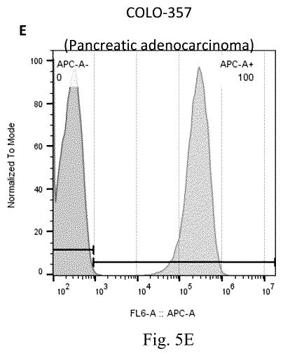

MUC1 is expressed on a vast variety of human cancer cell lines: A list of human cancer cell lines were screened for the expression of surface MUC1 using chimeric IgG1 anti-MUC1-SEA mAb as shown in FIGS. 4A-4D. Those cancer cell lines are then categorized as MUC1+, MUC1++ and MUC1+++ depending on the log shift of fluorescence for MUC1 as compared to the reference negative control. Of all the cell lines screened Colo357 and ZR75 were found to have the highest surface MUC1 expression and were denoted as MUC1+++; whereas HS766T, Jurkat and Raji were found to have no detectable surface MUC1. Post humanization of chimeric IgG1 anti-MUC1-SEA mAb, the humanized IgG1 anti-MUC1-SEA mAb (HM5, LM4) was also checked for the binding to the human cancer cell lines having varying expression of MUC1 as demonstrated in FIGS. 5A-5E.

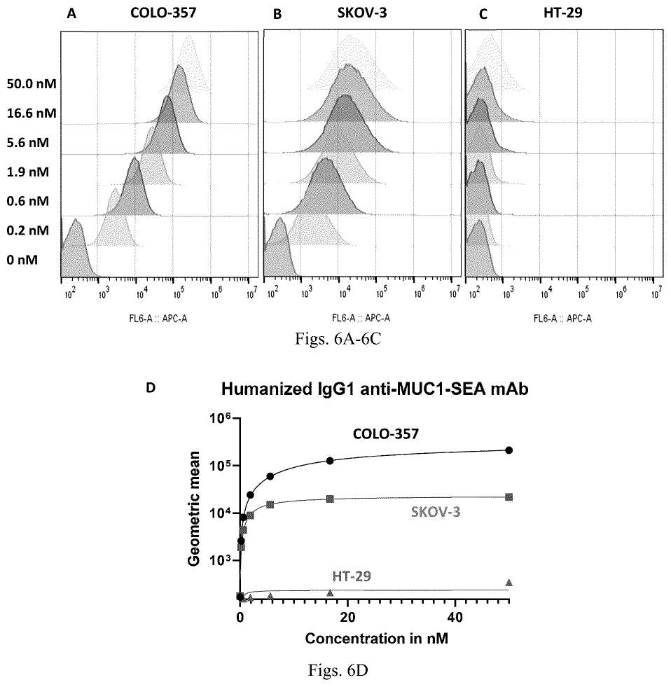

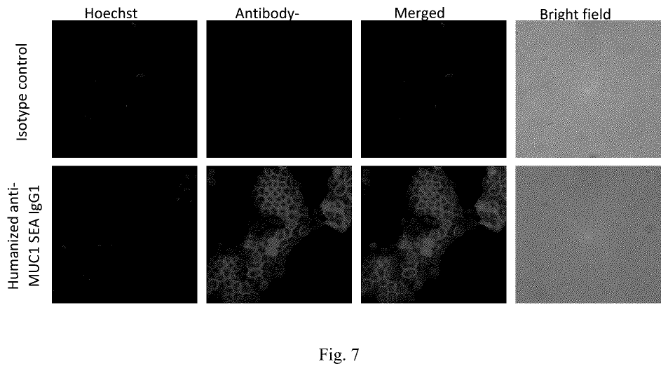

Dose dependent binding of Humanized IgG1 anti-MUC1-SEA mAb (HM5 LM4) on human carcinoma cell lines: Dose dependent binding of humanized IgG1 anti-MUC1-SEA mAb (HM5 LM4) with a range of 0 to 50 nM was performed on human carcinoma cell lines expressing high, moderate and low/no MUC1 using flow-cytometry as shown in FIGS. 6A-6C. Colo357 (MUC1+++ cell line), SKOV3 (MUC1++ cell line) and HT29 (MUC1−/+ cell line) exhibited dose dependent increase in the fluorescence signal associated with MUC1. The dose response plotted against mean fluorescence intensity is demonstrated in FIG. 6D.

Humanized IgG anti-MUC1-SEA mAb is internalized in MUC1 expressing cell line: ˜40% of humanized IgG1 anti-MUC1-SEA mAb (HM5 LM4) is internalized in MUC1-SEA expressing COLO357 cell line as shown in Table 4 below. The internalization has been further demonstrated using microscopic imaging of the MUC1-SEA expressing COLO357 cells with Alexa647 labelled humanized IgG anti-MUC1-SEA mAb as shown in FIG. 7. Surface as well as cytoplasmic staining by humanized anti-MUC1 SEA IgG1 (red) on MUC1 expressing COLO-357 cell line indicating the binding as well as the internalization.

TABLE 4

| | Percent Internalization | | | | |
|---|---|---|---|---|---|
| | 0 Hour | 0.5 Hour | 1 Hour | 2 Hour | 4 Hour |
| Humanized | 0 | 8.4 | 19.3 | 25.8 | 36.7 |
| anti-MUC1 | | | | | |
| SEA IgG1 | | | | | |

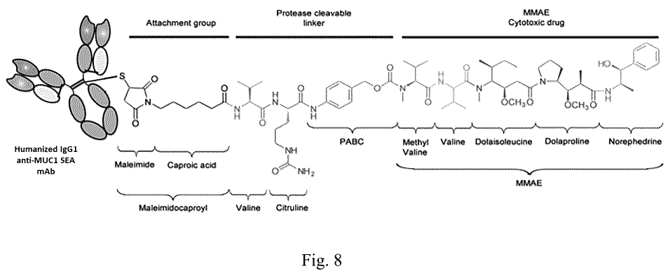

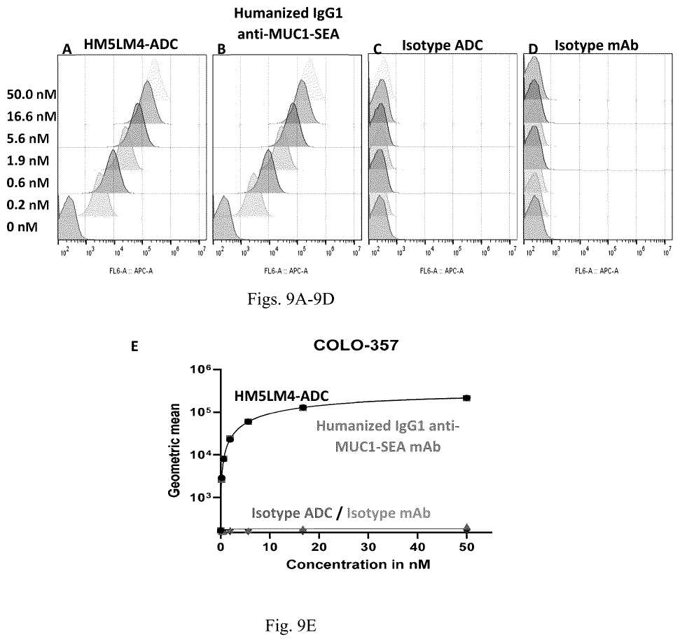

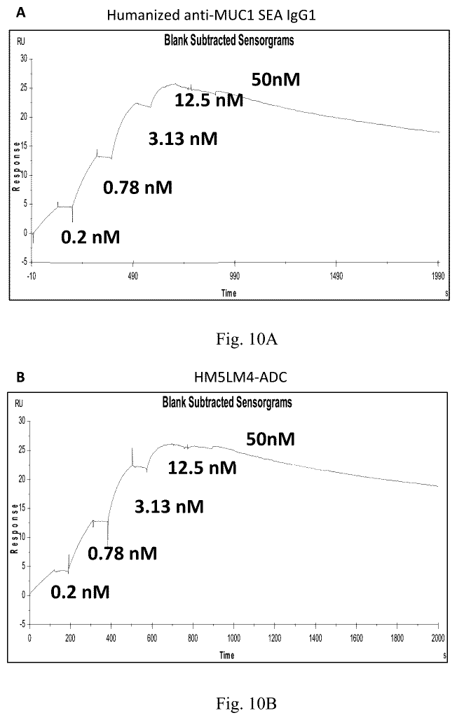

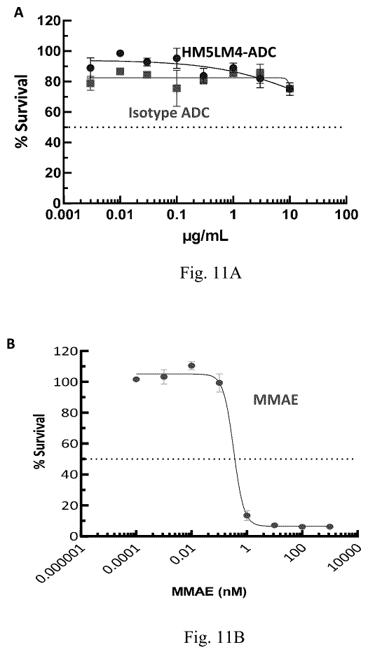

Binding strength of HM5LM4-ADC (humanized IgG anti-MUC1-SEA mAb conjugated to MC-Val-Cit-PABC-MMAE) is similar to that of unconjugated humanized IgG anti-MUC1-SEA mAb: Post conjugation of the MC-Val-Cit-PABC-MMAE linker-payload (FIG. 8) to humanized IgG1 anti-MUC1-SEA mAb to make the antibody drug conjugate (ADC) which is named as HM5LM4-ADC, the binding strength remained similar to that of the unconjugated parent antibody. This has been demonstrated by comparing the dose response for binding of HM5LM4-ADC and unconjugated antibody to COLO357 cells using flow cytometry (FIGS. 9A-9E). Similarly, it has been validated using SPR for the dissociation constant of HM5LM4-ADC and unconjugated antibody remains comparable i.e. ˜40 pM antigen as shown in FIGS. 10A and 10B and Table 5.

TABLE 5

| | ka | kd | KD |
|---|---|---|---|
| Antibody/ADC | (1/Ms) | (1/s) | (M) |
| Humanized anti- | 7.07E+06 | 2.81E−04 | 3.97E−11 |
| MUC1 SEA IgG1 | | | |
| HM5LM4-ADC | 6.12E+06 | 2.48E−04 | 4.06E−11 |

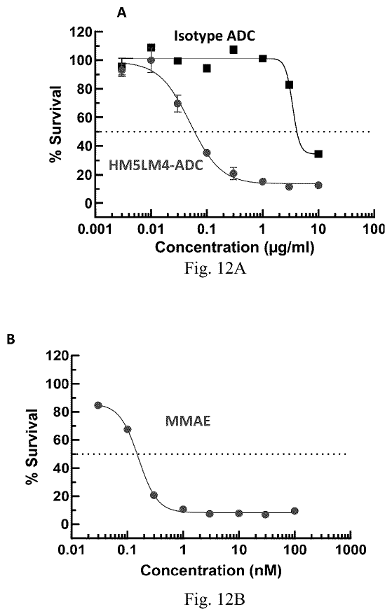

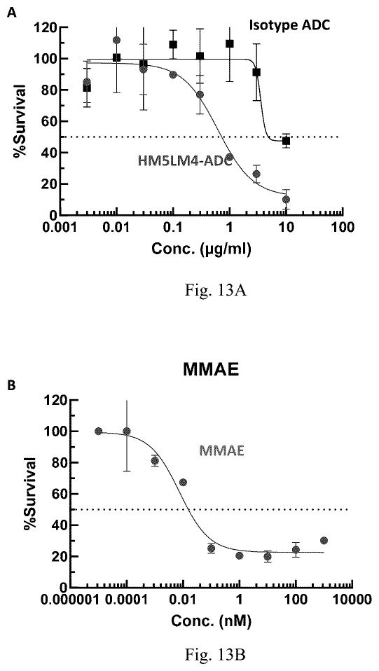

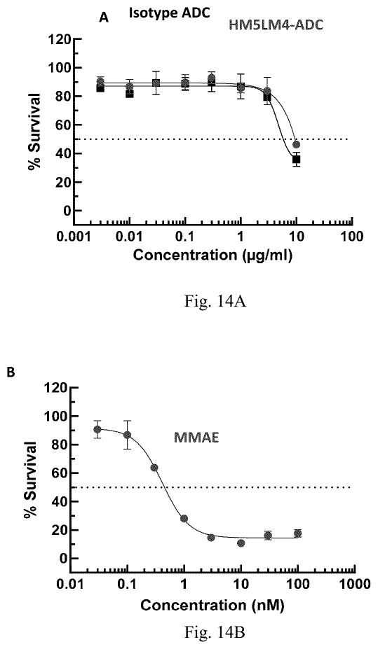

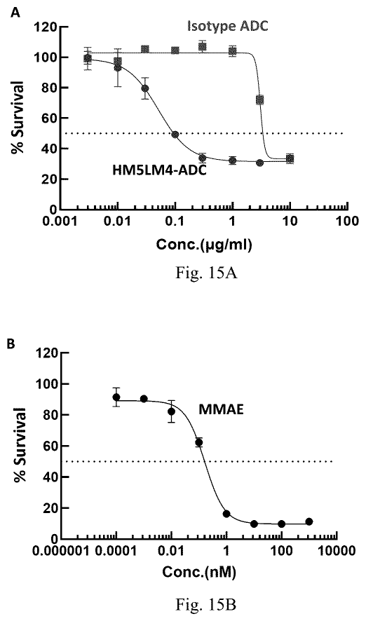

HM5LM4-ADC exerts its cytotoxic effect specifically on high MUC1 expressing cell lines: Human cancer cell lines exhibiting varying levels of the MUC1 were studies for the target specific cytotoxic effect of HM5LM4-ADC. Data demonstrated that in-vitro cytotoxicity of HM5LM4-ADC was only observed against high MUC1-SEA expressing COLO-357 cell line, BxPC3 cell line and T47D cell line (FIGS. 12A-12B, 13A-13B and 15A-15B). However, no cytotoxic effect was observed on no and moderate MUC1 expressing cell line, i.e., HT-29 (FIGS. 11A and 11B) and SKOV-3 (FIGS. 14A and 14B) cells, respectively.

Tables 6-8 below show the IC₅₀ on COLO-357 and BxPC3 pancreatic carcinoma cells and T47D breast carcinoma cells of HM5LM4-ADC and isotype ADC with its respective equivalent MMAE and free MMAE.

TABLE 6

| | | Equivalent |
|---|---|---|
| Treatment on | ADC IC₅₀ | MMAE IC₅₀ |
| COLO-357 | in μg/ml | in nM |
| HM5LM4-ADC | 0.05 | 1.01 |
| Isotype ADC | 3.45 | 60.97 |
| Free MMAE | — | 0.16 |

TABLE 7

| | | Equivalent |
|---|---|---|
| Treatment | ADC IC₅₀ | MMAE IC₅₀ |
| on BxPC3 | in μg/ml | in nM |
| HM5LM4-ADC | 0.62 | 12.57 |
| Isotype ADC | 3.6 | 73.00 |
| Free MMAE | — | 0.02 |

TABLE 8

| | | Equivalent |
|---|---|---|
| Treatment | ADC IC₅₀ | MMAE IC₅₀ |
| on T47D | in μg/ml | in nM |
| HM5LM4-ADC | 0.1 | 2.03 |
| Isotype ADC | No cytotoxic | 202.80- |
| | effect up | |
| | to 10 μg/ml | |
| Free MMAE | — | 0.07 |

### Example 4

### Efficacy of HM5LM4-ADC in Xenograft Mouse Models

To evaluate the anti-tumor efficacy of HM5LM4-ADC, human tumor xenograft mouse models were used. Human cancer cell lines suspended in a mixture of serum-free growth media and matrigel (1:1 ratio) were inoculated subcutaneously into the right flank region. After palpable tumors formed, animals were randomized into different groups to obtain a uniform distribution of animals in each group with comparable mean tumor volume. Animals were then administered a treatment. Throughout the study duration, animal handling and intravenous treatments were performed in an aseptic environment. During the study, data of tumor sizes, body weights and animal health signs were collected twice a week. Response to treatment was interpreted by calculating the tumor growth inhibition (TGI), tumor regression, partial or complete response, body weight changes and general health signs.

The percent TGI was computed using the formula: (1−(Treatment mm³_{final}−Treatment mm³ᵢₙᵢₜᵢₐₗ)/(Control mm³_{final}−Control mm³ᵢₙᵢₜᵢₐₗ))×100. The percent tumor regression was computed using the formula: ((Treatmentᵢₙᵢₜᵢₐₗ−Treatment_{final})/Treatmentᵢₙᵢₜᵢₐₗ)×100

The criteria for partial response (PR) was as follows: At least 30% reduction from initial tumor volume

The criteria for complete response (CR) was as follows: Complete disappearance of tumor

All values presented as mean±S.E.M. (standard error of mean) and the statistical measure of efficacy analyzed using the two-way ANOVA with Dunnett's multiple comparisons test using GraphPad Prism (version 9.3.1). The statistical data shown in the graphs is for the last time point.

### In Vivo Anti-Tumor Efficacy of HM5LM4-ADC in an Ovarian Cancer Xenograft Model in Mice

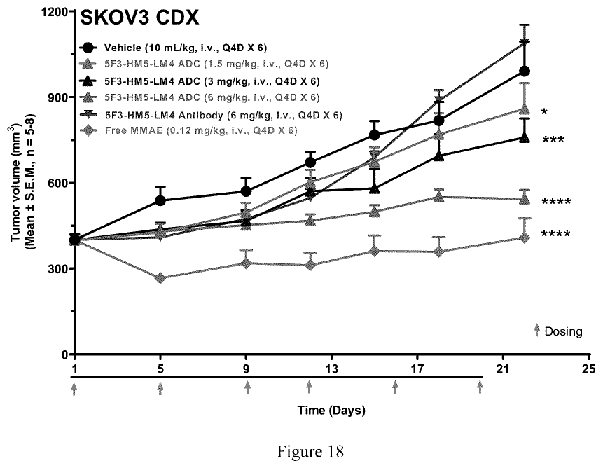

The anti-tumor efficacy of HM5LM4-ADC was evaluated in the MUC1 expressing ovarian cancer cell line SKOV3 derived xenograft model. Animals were supplemented with estrogen pellets implanted subcutaneously 3 days before cell inoculation. The subcutaneous tumor bearing female Balb/c athymic nude mice were administered intravenously with (a) 1.5, 3, 6 mg/kg of HM5LM4-ADC, (b) HM5-LM4 (5F3) antibody, or (c) 0.12 mg/kg free MMAE payload (equivalent to 6 mg/kg HM5LM4-ADC) once every four days with a total of six doses and monitored for the growth of tumors and health signs. The results are shown in FIG. 18. HM5LM4-ADC is referred to as 5F3-HM5-LM4 ADC in FIG. 18. The tumor growth inhibition (TGI) obtained for HM5LM4-ADC 6 mg/kg treatment was 99±5% (p<0.0001) and for 3 mg/kg it was 63±14% (p<0.0001) while, for the 1.5 mg/kg HM5LM4-ADC treatment, a marginal TGI of 45±17% (p<0.05) obtained. The free MMAE payload administered at 0.12 mg/kg also showed a TGI of 106±10% (p<0.0001). All dose regimens of vehicle and HM5LM4-ADC were tolerated well in the xenograft model; however, mortality was observed in 3 of 8 animals that received the free MMAE.

### In Vivo Anti-Tumor Efficacy of HM5LM4-ADC in a Pancreatic Cancer Xenograft Model in Mice

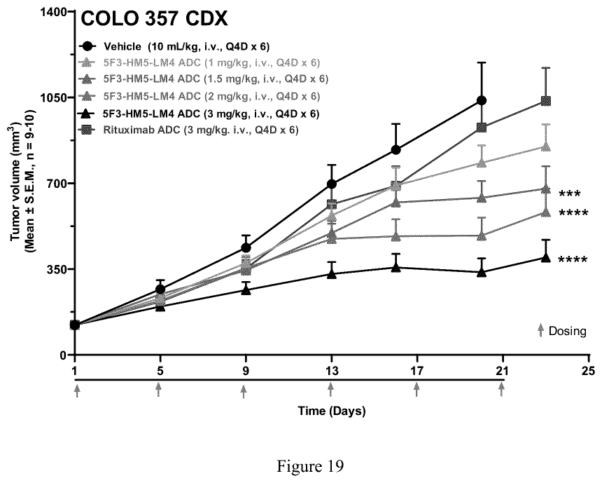

Anti-tumor efficacy of HM5LM4-ADC was evaluated in the MUC1 expressing pancreatic cancer cell line COLO 357 derived xenograft model. Subcutaneous tumor bearing female Balb/c athymic nude mice were administered intravenously with 1, 1.5, 2, or 3 mg/kg dose levels of HM5LM4-ADC or 3 mg/kg rituximab ADC (as a non-binding control) once every four days with a total of six doses and monitored for the growth of tumors and health signs. The results are shown in FIG. 19. The tumor growth inhibition (TGI) obtained for 3 mg/kg HM5LM4-ADC treatment was 76±6% (p<0.0001) and for 2 mg/kg it was 60±8% (p<0.0001) while, for the 1.5 mg/kg HM5LM4-ADC treatment, the TGI was 43±7% (p=0.001). The 3 mg/kg rituximab ADC was inefficacious (TGI=12±11). All dose regimens of vehicle and HM5LM4-ADC were tolerated well in the xenograft model.

### In Vivo Anti-Tumor Efficacy of HM5LM4-ADC in a Breast Cancer Xenograft Model in Mice

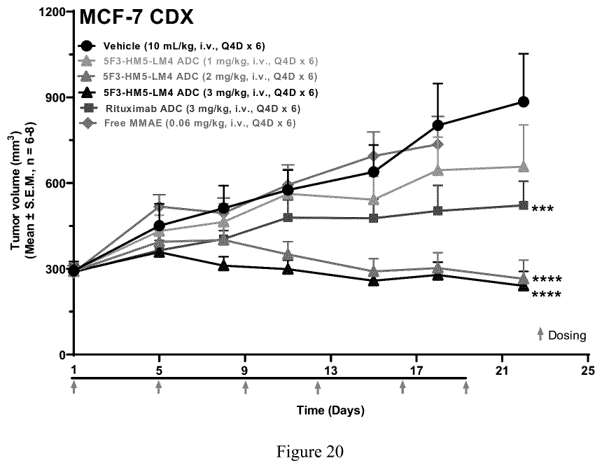

Anti-tumor efficacy of HM5LM4-ADC was evaluated in the MUC1 expressing breast cancer cell line MCF7 derived xenograft model. Animals were supplemented with estrogen pellets implanted subcutaneously 3 days before cell inoculation. The subcutaneous tumor bearing female Balb/c athymic nude mice were administered intravenously with 1, 2, 3 mg/kg of HM5LM4-ADC, 3 mg/kg rituximab ADC (as a non-binding control) or 0.06 mg/kg free MMAE payload once every four days with a total of six doses and monitored for the growth of tumors and health signs. The results are shown in FIG. 20. The tumor growth inhibition (TGI) obtained for 3 mg/kg HM5LM4-ADC treatment was 109±8% (p<0.0001) and for 2 mg/kg it was 104±9% (p<0.0001). The HM5LM4-ADC treatment also resulted in tumor regression of 12±20% in the 2 mg/kg group and 16±16 in the 3 mg/kg group. The rituximab ADC administered at 3 mg/kg also showed efficacy with TGI of 60±14% (p<0.001). In the free MMAE treated group, there was mortality in 3 of 8 animals on day 22. The 2 mg/kg HM5LM4-ADC regimen completely inhibited growth of MCF7 xenografts.

### In Vivo Anti-Tumor Efficacy of HM5LM4-ADC in a Head and Neck Xenograft Model in Mice

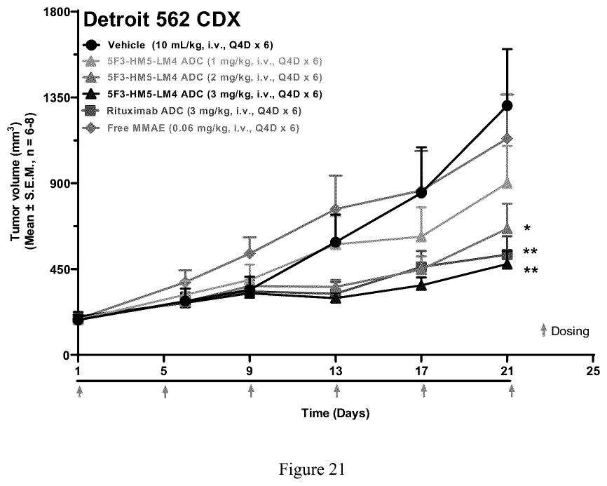

Anti-tumor efficacy of HM5LM4-ADC was evaluated in the MUC1 expressing head and neck cancer cell line Detroit 562 derived xenograft model. The subcutaneous tumor bearing female Balb/c athymic nude mice were administered intravenously with 1, 2, 3 mg/kg of HM5LM4-ADC, 3 mg/kg rituximab ADC (as a non-binding control) or 0.06 mg/kg free MMAE payload once every four days with a total of six doses and monitored for the growth of tumors and health signs. The results are shown in FIG. 21. The tumor growth inhibition (TGI) obtained for 3 mg/kg HM5LM4-ADC treatment was 72±5% (p<0.001) and for 2 mg/kg it was 60±11% (p<0.05). The rituximab ADC administered at 3 mg/kg also showed efficacy with a TGI of 70±7% (p<0.01). All dose regimens of vehicle and HM5LM4-ADC were tolerated well in the xenograft model.

### In Vivo Anti-Tumor Efficacy of HM5LM4-ADC in a Pancreatic Cancer Xenograft Model in Mice

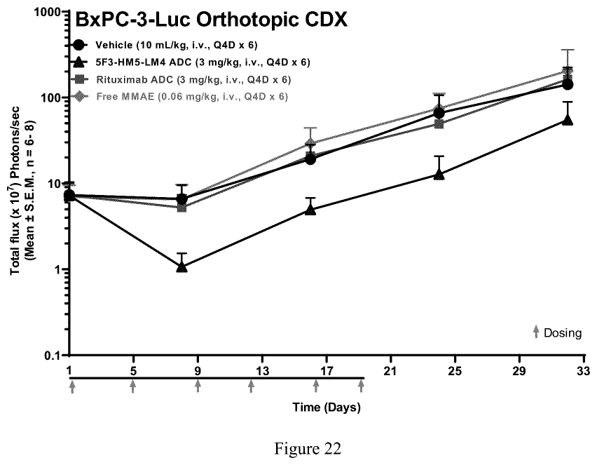

Anti-tumor efficacy of HM5LM4-ADC was evaluated in the MUC1 expressing cell line BxPc3-Luc derived xenograft model. The orthotopically established tumor bearing female Balb/c athymic nude mice were administered intravenously with 3 mg/kg HM5LM4-ADC, rituximab ADC (as a non-binding control) or equivalent free MMAE payload at 0.06 mg/kg once every four days with a total of six doses. Growth of tumors as a function of increase in bioluminescence imaging (BLI) signals measured in photons/see values were obtained by whole animal imaging in an IVIS imager within 15 minutes after injecting the D-luciferin substrate (150 mg/kg, i.p., single dose/mouse). The results are shown in FIG. 22. Among all groups, treatment with HM5LM4-ADC decreased tumor growth initially; however, tumor regrowth occurred with a marginal tumor growth inhibition (TGI) of 65±25%. All treatments were tolerated well in the orthotopic xenograft model.

### In Vivo Anti-Tumor Efficacy of HM5LM4-ADC in the Lung Cancer Xenograft Model in Mice

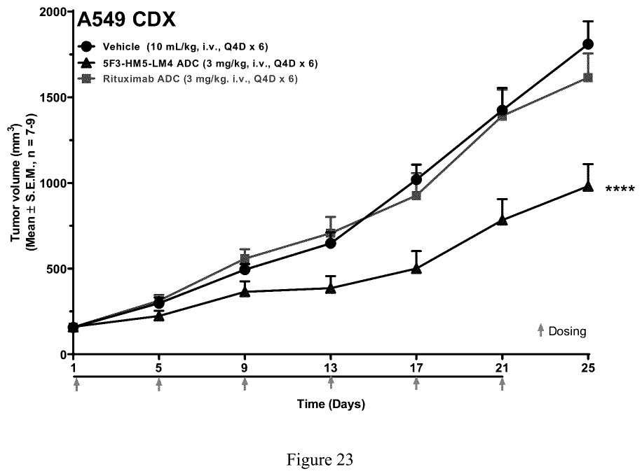

Anti-tumor efficacy of HM5LM4-ADC was evaluated in a MUC1 low expressing cell line A549 derived xenograft model. The subcutaneous tumor bearing male Balb/c athymic nude mice were administered intravenously with 3 mg/kg HM5LM4-ADC or rituximab ADC (as a non-binding control) once every four days with a total of six doses. The results are shown in FIG. 23. Treatment with HM5LM4-ADC resulted in a moderate tumor growth inhibition (TGI) of 50±7% (p<0.0001). All treatments were tolerated well in the xenograft model.

### Example 5

### Stability and Pharmacokinetics of HM5LM4-ADC

### Stability of HM5LM4-ADC in Mouse and Human Plasma

The stability of HM5LM4-ADC in mouse and human plasma was assessed by monitoring the release of MMAE. The HM5LM4-ADC was spiked to plasma at an MMAE equivalent concentration of 5000 nM and incubated at 37° C. for pre-defined time points up to 48 hours. At the end of 48 hours, the MMAE released in human plasma was <5 nM, while in mouse plasma it was 214 nM corresponding to <0.1% and 4.5% MMAE release in human and mouse plasma, respectively.

### Stability of HM5LM4-ADC in Purified Cathepsin B Enzyme Assay

The stability of the HM5LM4-ADC in purified Cathepsin B enzyme was assessed by monitoring the release of MMAE. The HM5LM4-ADC was incubated at an MMAE equivalent concentration of 5000 nM and incubated at 37° C. for pre-defined time points up to 6 hours. At the end of incubation, the MMAE released was 2800 nM, corresponding to 56% MMAE release. The data suggests cathepsin B mediated release of the payload by acting on the val-cit linkage.

### Pharmacokinetics of HM5LM4-ADC in Tumor Bearing Mice

1. Single dose pharmacokinetics study of HM5LM4-ADC in COLO357 Tumor Bearing Mouse by Intravenous and Intraperitoneal Administration

The objective of this study was to evaluate plasma and tumor pharmacokinetics of HM5LM4-ADC, total antibody (TAB) and unconjugated MMAE (uMMAE) following single intravenous administration at 10 mg/kg single intravenous (IV) or intraperitoneal (IP) administration of HM5LM4-ADC in a COLO357 tumor bearing mouse.

The blood samples were collected from the mouse at 0.017, 0.5, 1, 2, 4, 8, 24, 48, 72, 96, 168 and 336 hours post-dose (n=3 mice/time point; sparse sampling design) through retro-orbital plexuses and plasma was immediately harvested. At 24, 48, 72, 96, 168 and 336 hour time-points, the tumor was collected. Analysis of total antibody (TAB) and conjugated antibody (CAB) was done by a fit for purpose ELISA based method and analysis of free payload (MMAE) was done by a fit for purpose LC-MS/MS based method. Pharmacokinetic analysis was performed by the non-compartmental analysis tool of Phoenix WinNonlin® software. The results are shown in Tables 9 and 10 below.

TABLE 9

| | | | C₀ | Cₘₐₓ | Tₘₐₓ | AUCₗₐₛₜ | AUC_{INF} | T_{1/2} | |
|---|---|---|---|---|---|---|---|---|---|
| | | Dose | (nM) | (nM) | (h) | (h × *nM) | (h × *nM) | (h) | % F |
| Analyte | Route | (mg/kg) | Plasma | | | | | | |
| TAB | IP | 10 | | 761 | 8 | 67409 | 84403 | 155 | 224 |
| | IV | 10 | 813 | — | — | 31235 | 37755 | 135 | — |
| CAB | IP | 10 | | 491 | 8 | 39971 | 42580 | 84 | 183 |
| | IV | 10 | 664 | — | — | 22900 | 23268 | 57 | — |
| uMMAE | IP | 10 | — | 3.3 | 4 | 115 | 132 | 34 | 98 |
| | IV | 10 | 19 | — | — | 122 | 134 | 51 | — |

TABLE 10

| | | | | AUCₗₐₛₜ | AUC_{INF} | T_{1/2} | Tumor/ |
|---|---|---|---|---|---|---|---|
| | | Dose | Cₘₐₓ | (h* × | (h* × | | plasma |
| | | (mg/ | (nM) | nM) | nM) | (h) | AUC ratio |
| Analyte | Route | kg) | Tumor | | | | |
| TAB | IP | 10 | 25 | 5353 | NA | NA | 0.1 |
| | IV | 10 | 20 | 4847 | NA | NA | 0.2 |
| CAB | IP | 10 | 13 | 2437 | 4240 | 266 | 0.1 |
| | IV | 10 | 15 | 3156 | 3721 | 112 | 0.1 |
| uMMAE | IP | 10 | 299 | 42947 | 44127 | 59 | 374 |
| | IV | 10 | 334 | 48870 | 50009 | 63 | 401 |

NA=Not applicable

In general, both CAB and TAB exposure were ˜2-fold higher following intraperitoneal administration than intravenous administration. Tumor-exposure of MMAE was substantially higher as compared to plasma and comparable in both routes of administration.

### 2. Single Dose Pharmacokinetics Study of HM54LM4-ADC in COLO357 Tumor Bearing Mouse by Intravenous Administration

The objective of this study was to evaluate serum, tumor and liver pharmacokinetics of TAB, CAB and uMMAE following 3 mg/kg single IV administration of M5LM4-ADC and pharmacokinetics of MMAE following 0.06 mg/kg single IV administration of MMAE alone in a COLO357 tumor bearing mouse model.

The blood samples were collected from the mouse at 0.083, 0.5, 1, 2, 4, 8, 24, 48, 72, 96, 168 and 336 hours post-dose (n=3 mice/time point; sparse sampling design) through retro-orbital plexuses and serum was immediately harvested. At 24, 48, 72, 96, 168 and 336 hour time-points, the tumor and liver were collected. Analysis of total antibody (TAB) and conjugated antibody (CAB) was done by a fit for purpose ELISA based method and analysis of free payload (MMAE) was done by a fit for purpose LC-MS/MS based method. Pharmacokinetic analysis was performed by the non-compartmental analysis tool of Phoenix WinNonlin® software. The results are shown in Tables 11 and 12 below.

TABLE 11

| | | Serum | | | | | | | |
|---|---|---|---|---|---|---|---|---|---|
| | | Tₘₐₓ | Cₘₐₓ | AUC₀₋₂₄ | AUC₀₋₃₃₆ | AUC_{INF} | Vss | CL | T_{1/2} |
| Group ID | Analyte | (h) | (nM) | (h*nM) | (h*nM) | (h*nM) | (L/kg) | (mL/min/kg) | (h) |
| HM5LM4- | TAB | 0.5 | 360 | 4366 | 9624 | 9904 | 0.1 | 0.03 | 78 |
| ADC | | | | | | | | | |
| (3 mg/kg, | CAB | 0.5 | 416 | 4862 | 9196 | 9272 | NA | NA | 59 |
| IV) | uMMAE | 0.5 | 8 | 32 | 70 | 71 | NA | NA | 37 |
| MMAE | MMAE | 0.08 | 8 | 18 | 21 | 21 | 34 | 64 | 5 |
| (0.06 | | | | | | | | | |
| mg/kg, IV) | | | | | | | | | |

TABLE 12

| | | Tumor | | | | Liver | | | |
|---|---|---|---|---|---|---|---|---|---|
| | | Cₘₐₓ | AUC₀₋₃₃₆ | T_{1/2} | AUC | Cₘₐₓ | AUC₀₋₃₃₆ | T_{1/2} | AUC |
| Group ID | Analyte | (nM) | (h*nM) | (h) | ratio (Kp) | (nM) | (h*nM) | (h) | ratio (Kp) |
| HM5LM4- | TAB | 10 | 2275 | NR | 0.2 | 10 | 846 | 61 | 0.1 |
| ADC | CAB | 8.4 | 1996 | 191 | 0.2 | 2.6 | 228 | 59 | 0.02 |
| (3 mg/kg, | uMMAE | 84 | 16820 | 70 | 240 | 32 | 2686 | 56 | 38 |
| IV) | | | | | | | | | |
| MMAE | MMAE | 11 | 1391 | 154 | 66 | 1.5 | 190 | 182 | 9 |
| (0.06 mg/kg, | | | | | | | | | |
| IV) | | | | | | | | | |

Following a single intravenous administration of HM5LM4-ADC in mice, tumor exposure of uMMAE was 240-fold higher than in serum and 6.2-fold higher than in the liver. Following a single intravenous administration of MMAE in mice, tumor exposure was 66-fold higher than in serum and 7-fold higher than in the liver.

### 3. Single Dose Pharmacokinetics Study of HM5LM4-ADC in SKOV3 Tumor Bearing Mouse by Intravenous Administration

The objective of this study was to evaluate serum, tumor and tissue pharmacokinetics of TAB, CAB and unconjugated MMAE in a SKOV3 tumor bearing mouse model following single IV administration of HM5LM4-ADC at a dose of 3 mg/kg.

The blood samples were collected from the mice at 0.083, 0.5, 1, 2, 4, 24, 48, 72, 96 and 168 hours post-dose (n=3 mice/time point; sparse sampling design) through retro-orbital plexuses and serum was immediately harvested. At 24, 48, 72, 96 and 168 hour time-points, tumor, liver, brain, spleen, lungs, and kidney tissues were collected. Analysis of total antibody (TAB) and conjugated antibody (CAB) was done by a fit for purpose ELISA based method and analysis of free payload (MMAE) was done by a fit for purpose LC-MS/MS based method. Pharmacokinetic analysis was performed by the non-compartmental analysis tool of Phoenix WinNonlin® software. The results are shown in Tables 13 and 14 below.

TABLE 13

| | Serum | | | | | | | |
|---|---|---|---|---|---|---|---|---|
| | Tₘₐₓ | Cₘₐₓ | AUC₀₋₂₄ | AUC₀₋₁₆₈ | AUC_{INF} | Vss | CL | T_{1/2} |
| Analyte | (h) | (nM) | (h*nM) | (h*nM) | (h*nM) | (L/kg) | (mL/min/kg) | (h) |
| TAB | — | 721 | 8676 | 36226 | 45757 | 0.05 | 0.01 | 74 |
| CAB | — | 1058 | 11919 | 48969 | 50731 | NA | NA | 37 |
| uMMAE | 0.08 | 3 | 31 | 84 | 95 | NA | NA | 50 |

TABLE 14

| | Tumor | | | |
|---|---|---|---|---|
| | | | | AUC |
| | Cₘₐₓ | AUC₀₋₁₆₈ | T_{1/2} | ratio |
| Analyte | (nM) | (h*nM) | (h) | (Kp) |
| TAB | 63 | 3968 | 70 | 0.11 |
| CAB | 9.2 | 914 | 58 | 0.02 |
| uMMAE | 50 | 6001 | 105 | 71 |

Following a single intravenous administration of HM5LM4-ADC in mice, uMMAE exposure in tumor was 71-fold higher than serum and the descending order of, uMMAE tissue/plasma exposure ratio is: Tumor (71)>Liver (39)>Kidney (31)>Spleen (16)>Lung (10)>Brain (1.8).

Table A below provides the sequences referenced above. SEQ ID NOs: 1-15 are in the format FR1-CDR1-FR2-CDR2-FR3-CDR3-FR4. Bolding in the sequences indicates a CDR region. Underlining indicates a mutation.

TABLE A

| | SEQ ID | |
|---|---|---|
| Description | NO: | Sequence |
| m5F3_VH domain with the | 1 | DVQVQESGPD LVKPSQSLSL |
| broadest CDR defined | | TCTVTGHSIT RGSSWHWIRQ |
| (FR1-CDR1-FR2-CDR2- | | FPGNKLEWMG YIHYGGGTSY |
| FR3-CDR3-FR4) | | NPSLKSRISI TRDTSKNQFF |
| | | LQLNSVTTED TATFFCARYS |
| | | YDITYRWFFD VWGAGTTVIV SS |
| m5F3_VL domain with the | 2 | DILLTQSPAI LSVSPGERVS |
| broadest CDR defined | | FSCRASQNIG TSIHWYQQRK |
| (FR1-CDR1-FR2-CDR2- | | NGSPRLLIKY ASESISGIPS |
| FR3-CDR3-FR4) | | RFSGSGSGTD FTLSINSVES |
| | | EDMADYYCQQ NNNWPLTFGA GTKLELK |
| huCTM01_VH domain with | 3 | EIQLVQSGAE VKKPGSSVKV |
| the broadest CDR defined | | SCKASGYTFT DYYINWMRQA |
| (FR1-CDR1-FR2-CDR2- | | PGQGLEWIGW IDPGSGNTKY |
| FR3-CDR3-FR4) | | NEKFKGRATL TVDTSTNTAY |
| | | MELSSLRSED TAFYFCAREK |
| | | TTYYYAMDYW GQGTLVTVSS |
| huCTM01_VL domain with | 4 | DIQMTQSPST LSASVGDRVT |
| the broadest CDR defined | | ITCRSSKSLL HSNGDTFLYW |
| (FR1-CDR1-FR2-CDR2- | | FQQKPGKAPK LLMYRMSNLA |
| FR3-CDR3-FR4) | | SGVPSRFSGS GSGTEFTLTI |
| | | SSLQPDDFAT YYCMQHLEYP |
| | | FTFGQGTKVE VK |
| VH domain of SP66 | 5 | EIQLVQSGAE VKKPGSSVKV |
| (FR1-CDR1-FR2-CDR2- | | SCKASGHSIT RGSSWHWMRQ |
| FR3-CDR3-FR4) | | APGQGLEWIG YIHYGGGTSY |
| | | NPSLKSRATL TVDTSTNTAY |
| | | MELSSLRSED TAFYFCARYS |
| | | YDITYRWFFD VWGQGTLVTV SS |
| VL domain of SP66 | 6 | DIQMTQSPST LSASVGDRVT |
| (FR1-CDR1-FR2-CDR2- | | ITCRASQNIG TSIHWFQQKP |
| FR3-CDR3-FR4) | | GKAPKLLMYR ASESISGVPS |
| | | RFSGSGSGTE FTLTISSLQP |
| | | DDFATYYCQQ NNNWPLTFGQ GTKVEVK |
| VH domain of HM1 | 7 | EIQLVQSGAE VKKPGSSVKV |
| (The mutated residues are | | SCKVTGHSIT RGSSWHWMRQ |
| underlined) | | APGQGLEWIG YIHYGGGTSY |
| (FR1-CDR1-FR2-CDR2- | | NPSLKSRATL TVDTSTNTAY |
| FR3-CDR3-FR4) | | MELSSLRSED TAFYFCARYS |
| | | YDITYRWFFD VWGQGTLVTV SS |
| VH domain of HM2 | 8 | EIQLVQSGAE VKKPGSSVKV |
| (The mutated residues are | | SCKVTGHSIT RGSSWHWMRQ |
| underlined) | | APGQGLEWIG YIHYGGGTSY |
| (FR1-CDR1-FR2-CDR2- | | NPSLKSRITI TRDTSTNTAY |
| FR3-CDR3-FR4) | | MELSSLRSED TAFYFCARYS |
| | | YDITYRWFFD VWGQGTLVTV SS |
| VH domain of HM3 | 9 | EIQLVQSGAE VKKPGSSVKV |
| (The mutated residues are | | SCKVTGHSIT RGSSWHWMRQ |
| underlined) | | APGQGLEWIG YIHYGGGTSY |
| (FR1-CDR1-FR2-CDR2- | | NPSLKSRATL TRDTSTNTAY |
| FR3-CDR3-FR4) | | MELSSLRSED TAFYFCARYS |
| | | YDITYRWFFD VWGQGTLVTV SS |
| VH domain of HM4 | 10 | EIQLVQSGAE VKKPGSSVKV |
| (The mutated residues are | | SCKVTGHSIT RGSSWHWMRQ |
| underlined) | | APGQGLEWIG YIHYGGGTSY |
| (FR1-CDR1-FR2-CDR2- | | NPSLKSRATL TRDTSTNTFY |
| FR3-CDR3-FR4) | | MELSSLRSED TAFYFCARYS |
| | | YDITYRWFFD VWGQGTLVTV SS |
| VH domain of HM5 | 11 | EIQLVQSGAE VKKPGSSVKV |
| (The mutated residues are | | SCKVTGHSIT RGSSWHWMRQ |
| underlined) | | APGQGLEWIG YIHYGGGTSY |
| (FR1-CDR1-FR2-CDR2- | | NPSLKSRITI TRDTSTNTFY |
| FR3-CDR3-FR4) | | MELSSLRSED TAFYFCARYS |
| | | YDITYRWFFD VWGQGTLVTV SS |
| VL domain of LM1 | 12 | DIQMTQSPST LSASVGDRVT |
| (The mutated residues are | | ITCRASQNIG TSIHWFQQKP |
| underlined) | | GKAPKLLIKR ASESISGVPS |
| (FR1-CDR1-FR2-CDR2- | | RFSGSGSGTE FTLTISSLQP |
| FR3-CDR3-FR4) | | DDFATYYCQQ NNNWPLTFGQ GTKVEVK |
| VL domain of LM2 | 13 | DIQMTQSPST LSASVGDRVT |
| (The mutated residues are | | ITCRASQNIG TSIHWYQQKP |
| underlined) | | GKAPKLLIKR ASESISGVPS |
| (FR1-CDR1-FR2-CDR2- | | RFSGSGSGTE FTLTISSLQP |
| FR3-CDR3-FR4) | | DDFATYYCQQ NNNWPLTFGQ GTKVEVK |
| VL domain of LM3 | 14 | DIQMTQSPST LSASVGDRVT |
| (The mutated residues are | | ITCRASQNIG TSIHWYQQKP |
| underlined) | | GKAPKLLIKY ASESISGVPS |
| (FR1-CDR1-FR2-CDR2- | | RFSGSGSGTE FTLTISSLQP |
| FR3-CDR3-FR4) | | DDFATYYCQQ NNNWPLTFGQ GTKVEVK |
| VL domain of LM4 | 15 | DIQMTQSPST LSASVGDRVT |
| (The mutated residues are | | ITCRASQNIG TSIHWYQQKP |
| underlined) | | GKAPKLLIKY ASESISGVPS |
| (FR1-CDR1-FR2-CDR2- | | RFSGSGSGTE FTLTISSLQP |
| FR3-CDR3-FR4) | | DDFATYYCQQ NNNWPLTFGQ GTKVEIK |
| SP190-FP | 16 | CGCCACCGGC GCGCATTCT |
| SP191-RP | 17 | GGTGCTAGCG CTGGACACGG TCACCAG |
| SP192-FP | 18 | CACAGGCGCG CACTCCGACA |
| | | TCCAGATGAC ACAGAG |
| SP193-RP | 19 | GCCACCGTAC GCTTCACCTC |
| | | CACCTTGGTG |
| SP223-FP | 20 | GTCTTGCAAG GTGACAGGCC |
| | | ACTCCATCAC AAG |
| SP224-RP | 21 | ACCTTCACGC TGGAGCCT |
| SP227-FP | 22 | CACAAGGGAC ACCAGCACAA ACACCGCT |
| SP228-RP | 23 | ATGGTGATCC TAGACTTCAG |
| | | GCTTGGATTG TAG |
| SP229-FP | 24 | CACCCTGACA AGGGACACCA GCAC |
| SP230-RP | 25 | GCCCTAGACT TCAGGCTT |
| SP231-FP | 26 | CACAAACACC TTTTATATGG |
| | | AGCTGAGCTC |
| SP233-RP | 27 | CTGGTGTCCC TTGTCAGG |
| SP270-RP | 28 | CTGGTGTCCC TTGTGATGG |
| SP239-FP | 29 | TAAGCTGCTG ATCAAGAGAG |
| | | CTTCTGAGTC CATCAGC |
| SP240-RP | 30 | GGGGCCTTGC CGGGCTTC |
| SP237-FP | 31 | CATCCATTGG TACCAGCAGA AGC |
| SP238-RP | 32 | CTGGTGCCGA TATTCTGG |
| SP268-FP | 33 | GCTGATCAAG TACGCTTCTG |
| | | AGTCCATCAG CGG |
| SP269-RP | 34 | AGCTTAGGGG CCTTGCCG |
| SP332-FP | 35 | CAAGGTGGAG ATCAAGCGTA |
| | | CGGTGGCTGC AC |
| SP333-RP | 36 | GTGCCCTGGC CAAATGTCAG G |
| Heavy chain of HM5-LM4 | 37 | EIQLVQSGAE VKKPGSSVKV |
| (VH domain-CH domain) | | SCKVTGHSIT RGSSWHWMRQ |
| | | APGQGLEWIG YIHYGGGTSY |
| | | NPSLKSRITI TRDTSTNTFY |
| | | MELSSLRSED TAFYFCARYS |
| | | YDITYRWFFD VWGQGTLVTV |
| | | SSASTKGPSV FPLAPSSKST |
| | | SGGTAALGCL VKDYFPEPVT |
| | | VSWNSGALTS GVHTFPAVLQ |
| | | SSGLYSLSSV VTVPSSSLGT |
| | | QTYICNVNHK PSNTKVDKRV |
| | | EPKSCDKTHT CPPCPAPELL |
| | | GGPSVFLFPP KPKDTLMISR |
| | | TPEVTCVVVD VSHEDPEVKF |
| | | NWYVDGVEVH NAKTKPREEQ |
| | | YNSTYRVVSV LTVLHQDWLN |
| | | GKEYKCKVSN KALPAPIEKT |
| | | ISKAKGQPRE PQVYTLPPSR |
| | | EEMTKNQVSL TCLVKGFYPS |
| | | DIAVEWESNG QPENNYKTTP |
| | | PVLDSDGSFF LYSKLTVDKS |
| | | RWQQGNVFSC SVMHEALHNH |
| | | YTQKSLSLSP GK |
| Light chain of HM5-LM4 | 38 | DIQMTQSPST LSASVGDRVT |
| (VL domain-CL domain) | | ITCRASQNIG TSIHWYQQKP |
| | | GKAPKLLIKY ASESISGVPS |
| | | RFSGSGSGTE FTLTISSLQP |
| | | DDFATYYCQQ NNNWPLTFGQ |
| | | GTKVEIKRTV AAPSVFIFPP |
| | | SDEQLKSGTA SVVCLLNNFY |
| | | PREAKVQWKV DNALQSGNSQ |
| | | ESVTEQDSKD STYSLSSTLT |
| | | LSKADYEKHK VYACEVTHQG |
| | | LSSPVTKSFN RGEC |
| CH domain | 39 | ASTKGPSVFP LAPSSKSTSG |
| | | GTAALGCLVK DYFPEPVTVS |
| | | WNSGALTSGV HTFPAVLQSS |
| | | GLYSLSSVVT VPSSSLGTQT |
| | | YICNVNHKPS NTKVDKRVEP |
| | | KSCDKTHTCP PCPAPELLGG |
| | | PSVFLFPPKP KDTLMISRTP |
| | | EVTCVVVDVS HEDPEVKFNW |
| | | YVDGVEVHNA KTKPREEQYN |
| | | STYRVVSVLT VLHQDWLNGK |
| | | EYKCKVSNKA LPAPIEKTIS |
| | | KAKGQPREPQ VYTLPPSREE |
| | | MTKNQVSLTC LVKGFYPSDI |
| | | AVEWESNGQP ENNYKTTPPV |
| | | LDSDGSFFLY SKLTVDKSRW |
| | | QQGNVFSCSV MHEALHNHYT |
| | | QKSLSLSPGK |
| CL domain | 40 | RTVAAPSVFI FPPSDEQLKS |
| | | GTASVVCLLN NFYPREAKVQ |
| | | WKVDNALQSG NSQESVTEQD |
| | | SKDSTYSLSS TLTLSKADYE |
| | | KHKVYACEVT HQGLSSPVTK SFNRGEC |
| Human MUC1 | 41 | MTPGTQSPFF LLLLLTVLTV |
| (UniProt P15941) | | VTGSGHASST PGGEKETSAT |
| | | QRSSVPSSTE KNAVSMTSSV |
| | | LSSHSPGSGS STTQGQDVTL |
| | | APATEPASGS AATWGQDVTS |
| | | VPVTRPALGS TTPPAHDVTS |
| | | APDNKPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDTRPAPGS TAPPAHGVTS |
| | | APDNRPALGS TAPPVHNVTS |
| | | ASGSASGSAS TLVHNGTSAR |
| | | ATTTPASKST PFSIPSHHSD |
| | | TPTTLASHST KTDASSTHHS |
| | | SVPPLTSSNH STSPQLSTGV |
| | | SFFFLSFHIS NLQFNSSLED |
| | | PSTDYYQELQ RDISEMFLQI |
| | | YKQGGFLGLS NIKFRPGSVV |
| | | VQLTLAFREG TINVHDVETQ |
| | | FNQYKTEAAS RYNLTISDVS |
| | | VSDVPFPFSA QSGAGVPGWG |
| | | IALLVLVCVL VALAIVYLIA |
| | | LAVCQCRRKN YGQLDIFPAR |
| | | DTYHPMSEYP TYHTHGRYVP |
| | | PSSTDRSPYE KVSAGNGGSS |
| | | LSYTNPAVAA TSANL |
| DMB5F3 (5F3) heavy chain | 42 | MRVLILLCLF TAFPGVLSDV |
| variable region | | QVQESGPDLV KPSQSLSLTC |
| | | TVTGHSITRG SSWHWIRQFP |
| | | GNKLEWMGYI HYGGGTSYNP |
| | | SLKSRISITR DTSKNQFFLQ |
| | | LNSVTTEDTA TFFCARYSYD |
| | | ITYRWFFDVW GAGTTVIVSS |
| DMB5F3 (5F3) light chain | 43 | MVSTPQFLVF LLFWIPASRG |
| variable region | | DILLTQSPAI LSVSPGERVS |
| | | FSCRASQNIG TSIHWYQQRK |
| | | NGSPRLLIKY ASESISGIPS |
| | | RFSGSGSGTD FTLSINSVES |
| | | EDMADYYCQQ NNNWPLTFGA GTKLELK |
| SP291-FP | 44 | GTCTTGCAAG GCTTCTGGCC |
| | | ACTCCATCAC AAG |
| VH domain of HM6 | 45 | EIQLVQSGAE VKKPGSSVKV |
| (The mutated residues are | | SCKASGHSIT RGSSWHWMRQ |
| underlined) | | APGQGLEWIG YIHYGGGTSY |
| (FR1-CDR1-FR2-CDR2- | | NPSLKSRATL TRDTSTNTFY |
| FR3-CDR3-FR4) | | MELSSLRSED TAFYFCARYS |
| | | YDITYRWFFD VWGQGTLVTV SS |
| VH domain of HM7 | 46 | EIQLVQSGAE VKKPGSSVKV |
| (The mutated residues are | | SCKASGHSIT RGSSWHWMRQ |
| underlined) | | APGQGLEWIG YIHYGGGTSY |
| (FR1-CDR1-FR2-CDR2- | | NPSLKSRITI TRDTSTNTFY |
| FR3-CDR3-FR4) | | MELSSLRSED TAFYFCARYS |
| | | YDITYRWFFD VWGQGTLVTV SS |

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

1. A humanized monoclonal antibody which binds to the MUC1 SEA domain, wherein the antibody comprises:
(a) a heavy chain variable region comprising the amino acid sequence of any one of SEQ ID NOs. 7-11, 45, and 46; and
(b) a light chain variable region comprising the amino acid sequence of any one of SEQ ID NOs. 12-15.

2. The humanized antibody of claim 1, where the antibody comprises:
(a) a heavy chain variable region comprising the amino acid sequence of SEQ ID NO: 8 and a light chain variable region comprising the amino acid sequence of SEQ ID NO: 12;
(b) a heavy chain variable region comprising the amino acid sequence of SEQ ID NO: 8 and a light chain variable region comprising the amino acid sequence of SEQ ID NO: 13;
(c) a heavy chain variable region comprising the amino acid sequence of SEQ ID NO: 8 and a light chain variable region comprising the amino acid sequence of SEQ ID NO: 14;
(d) a heavy chain variable region comprising the amino acid sequence of SEQ ID NO: 9 and a light chain variable region comprising the amino acid sequence of SEQ ID NO: 13;
(e) a heavy chain variable region comprising the amino acid sequence of SEQ ID NO: 9 and a light chain variable region comprising the amino acid sequence of SEQ ID NO: 14;
(f) a heavy chain variable region comprising the amino acid sequence of SEQ ID NO: 10 and a light chain variable region comprising the amino acid sequence of SEQ ID NO: 13;
(g) a heavy chain variable region comprising the amino acid sequence of SEQ ID NO: 10 and a light chain variable region comprising the amino acid sequence of SEQ ID NO: 14;
(h) a heavy chain variable region comprising the amino acid sequence of SEQ ID NO: 11 and a light chain variable region comprising the amino acid sequence of SEQ ID NO: 13;
(i) a heavy chain variable region comprising the amino acid sequence of SEQ ID NO: 11 and a light chain variable region comprising the amino acid sequence of SEQ ID NO: 14; or
(j) a heavy chain variable region comprising the amino acid sequence of SEQ ID NO: 11 and a light chain variable region comprising the amino acid sequence of SEQ ID NO: 15.

3. The humanized antibody of claim 1, wherein the constant domain of the heavy chain comprises the amino acid sequence of SEQ ID NO: 39 and the constant domain of the light chain comprises the amino acid sequence of SEQ ID NO: 40.

4. A humanized monoclonal antibody which binds to the MUC1 SEA domain, wherein the antibody comprises a heavy chain variable region comprising the amino acid sequence of SEQ ID NO: 11 and a light chain variable region comprising the amino acid sequence of SEQ ID NO: 15.

5. A humanized monoclonal antibody which binds to the MUC1 SEA domain, wherein the antibody comprises a heavy chain comprising the amino acid sequence of SEQ ID NO: 37 and a light chain comprising the amino acid sequence of SEQ ID NO: 38.

6. The humanized antibody of claim 5, wherein the moiety 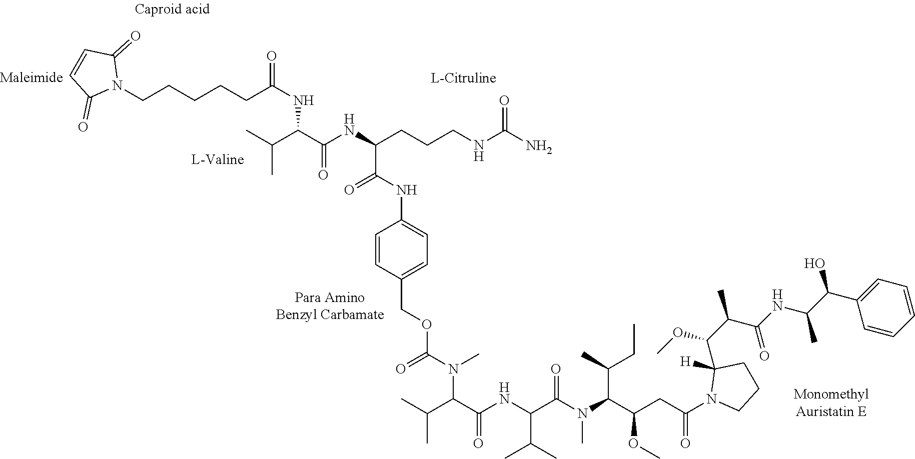 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)C(NC(=O)C(C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]
is conjugated through its maleimide terminus to the light chain of the humanized antibody.

7. (canceled)

8. The humanized monoclonal antibody of claim 1, wherein the antigen-binding fragment thereof is Fv, single chain Fv (scFv), single chain Fv-Fc (scFv-Fc), Fab′, Fab, F(ab′)₂ or F(ab)₂.

9. A humanized antibody that binds to an epitope in the MUC1 SEA domain, with a binding affinity K_{D} less than 100 pM.

10. The humanized antibody of claim 9, wherein the humanized antibody comprises a means for binding an epitope in the SEA domain of MUC1 (SEQ ID NO: 41) formed from arginine at position 1108, glutamic acid at position 1109, asparagine at position 1113 and glutamic acid at position 1118 of MUC1.

11. The humanized antibody of claim 9, wherein the humanized antibody binds to the same epitope as a chimeric antibody comprising a heavy chain variable region and a light chain variable region comprising the amino acid sequences of SEQ ID NOs: 1 and 2, respectively.

12. The humanized antibody of claim 9, wherein the antibody is a humanized form of any one of chimeric antibodies DMB4F4 (4F4), DMB7F3 (7F3), or DMB10F10 (10F10).

13. A humanized antibody that binds to an epitope in the SEA domain of MUC1 (SEQ ID NO: 41) formed from arginine at position 1108, glutamic acid at position 1109, asparagine at position 1113 and glutamic acid at position 1118 of MUC1.

14. (canceled)

15. An isolated nucleic acid molecule comprising a nucleotide sequence encoding an antibody of claim 1.

16. An expression vector comprising the isolated nucleic acid molecule of claim 15.

17. A host cell transfected with the expression vector according to claim 16.

18. An immunoconjugate comprising the antibody of claim 1 and an additional cytotoxic or therapeutic agent.

19. The immunoconjugate according to claim 18, wherein said cytotoxic agent is selected from a group consisting of alkylating drugs, anthracyclines, pyrimidine derivatives, vinca alkaloids, photodynamic drugs, platinum-containing compounds, taxanes, topoisomerase inhibitors, ribosome inactivating agents, agents that induce DNA damage, tubulin inhibitors, anti-mitotic agents, radioisotopes, cytotoxic antibodies and bacterial toxins.

20. The immunoconjugate of claim 18, wherein said cytotoxic agent is pseudomonas exotoxin.

21. The immunoconjugate of claim 18, wherein said cytotoxic agent is monomethyl auristatin E (MMAE).

22. The immunoconjugate of claim 21, wherein the antibody is conjugated to maleimidocaproyl-Val-Cit-PABC-MMAE.

23. An immunoconjugate comprising a humanized monoclonal antibody conjugated to maleimidocaproyl-Val-Cit-PABC-MMAE, where the antibody (i) binds to the MUC1 SEA domain and (ii) comprises a heavy chain comprising the amino acid sequence of SEQ ID NO: 37 and a light chain comprising the amino acid sequence of SEQ ID NO: 38.

24. The immunoconjugate according to claim 18, wherein said immunoconjugate reduces tumor volume upon administration to a subject with cancer.

25. A pharmaceutical composition comprising (a) the humanized monoclonal antibody of claim 1, the immunoconjugate of any one of claims 18-24, and (b) a pharmaceutically acceptable carrier, excipient, or diluent.

26. The pharmaceutical composition according to claim 25, wherein said pharmaceutical composition further comprises an additional therapeutic agent.

27. A method of treatment or amelioration of a disease or disorder comprising administering to a subject in need thereof a therapeutically effective amount of at least one humanized monoclonal antibody of claim 1.

28. The method of claim 27, wherein the disease or disorder is cancer.

29. The method of claim 28, wherein the cancer is a MUC1 expressing cancer.

30. The method of claim 28, wherein the cancer is selected from lung carcinoma, prostate carcinoma, breast carcinoma, ovarian carcinoma, colon carcinoma, pancreatic carcinoma, multiple myeloma, and acute myelogenous leukemia.

31. The method of claim 27, wherein the disease or disorder is an autoimmune or an inflammatory disease.

32. The method of claim 31, wherein the autoimmune or inflammatory disease is selected from rheumatoid arthritis, psoriatic arthritis, systemic lupus erythematosus, amyloidosis, and autoimmune pancreatitis.

33. The method of claim 27, wherein the disease or disorder is a non-malignant clinically significant abnormal growth condition.

34. The method of claim 27, wherein the method further comprises administering to a subject in need thereof an additional therapeutic agent.

35-45. (canceled)
